(19) United States
(12) Patent Application Publication
(10) Pub. No.: US 2019/0177317 A1
(43) Pub. Date: Jun. 13, 2019

(54) PROCESS FOR THE PREPARATION OF VENETOCLAX

(71) Applicant: MYLAN LABORATORIES LIMITED, HYDERABAD (IN)

(72) Inventors: RAJESH JOSHI, HYDERABAD (IN); ANIL KUMAR TRIPATHI, HYDERABAD (IN); CHANDRAKANT CHAUDHARI, HYDERABAD (IN); NAGARAJU GOTTUMUKKALA, HYDERABAD (IN); KIRAN POKHARKAR, HYDERABAD (IN); YOGESH SANGVIKAR, HYDERABAD (IN); LAKSHMANARAO VADALI, HYDERABAD (IN); SURESH BABU JAYACHANDRA, HYDERABAD (IN)

(73) Assignee: MYLAN LABORATORIES LIMITED, HYDERABAD (IN)

(21) Appl. No.: 16/324,614

(22) PCT Filed: Aug. 11, 2017
(86) PCT No.: PCT/IN2017/050341

§ 371 Date: Feb. 11, 2019

(30) Foreign Application Priority Data
Aug. 12, 2016 (IN) 201641027658
Sep. 23, 2016 (IN) 201641032593

(51) Int. Cl. C07D 471/04

(52) U.S. Cl. CPC C07D 471/04; A61P 35/02; C07B 2200/13

(57) ABSTRACT

The present disclosure provides novel synthetic process for the preparation of venetoclax. The disclosed processes involve the use of novel intermediates. Processes for the preparation of these intermediates are also disclosed as well as methods for the preparation of particularly useful salts thereof.

### CROSS-REFERENCE TO RELATED APPLICATIONS

This application claims the benefit of earlier Indian provisional patent application No. 201641027658 filed on Aug. 12, 2016 and Indian provisional patent application No. 201641032593 filed on Sep. 23, 2016 which is incorporated by reference herein in its entirety.

### BACKGROUND OF THE INVENTION

### Field of the Invention

The present invention relates generally to pharmaceutical active ingredients and methods for the preparation thereof. More specifically, the present invention provides processes for preparation of venetoclax and its pharmaceutically acceptable salts.

### Description of Related Art

Venetoclax, also known in the art as GDC-0199, ABT-199, or RG7601, is a BCL-2 inhibitor.

Venetoclax is chemically known as 4-[4-[[2-(4-chlorophenyl)-4,4-dimethylcyclohexen-1-yl]methyl]piperazin-1-yl]-N-[3-nitro-4-(oxan-4-ylmethylamino)phenyl]sulfonyl-2-(1H-pyrrolo[2,3-b]pyridin-5-yloxy)benzamide and has a structure as represented below in Formula-I:

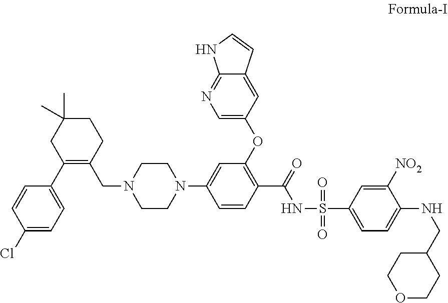 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is marketed in the United States under the brand name VENCLEXTA™ by AbbVie, Inc., and is indicated for the treatment of chronic lymphocytic leukemia.

Venetoclax and processes for the preparation thereof are disclosed in U.S. Pat. No. 8,546,399.

### SUMMARY OF THE INVENTION

In one aspect, the present invention provides a process for the preparation of venetoclax. In one embodiment, venetoclax may be prepared by a process that includes the steps of:
a. condensing formula 5 with formula 4 in the presence of a base to obtain formula 3

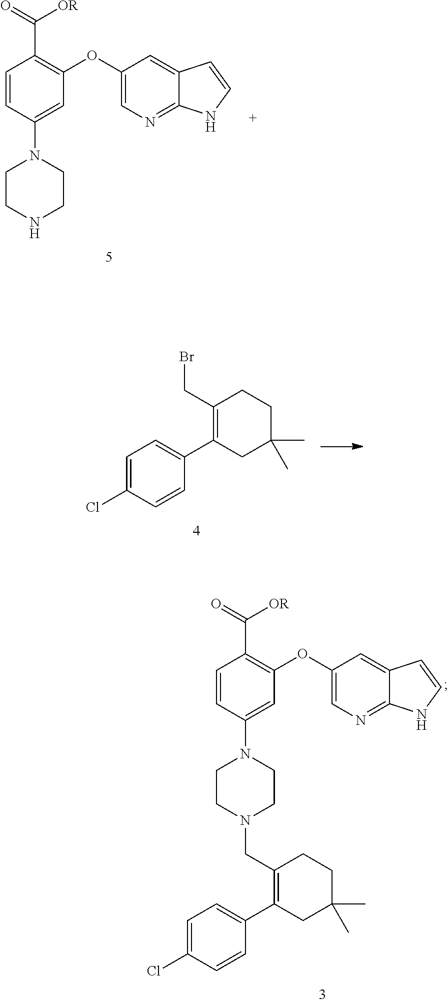

and
b. hydrolyzing formula 3

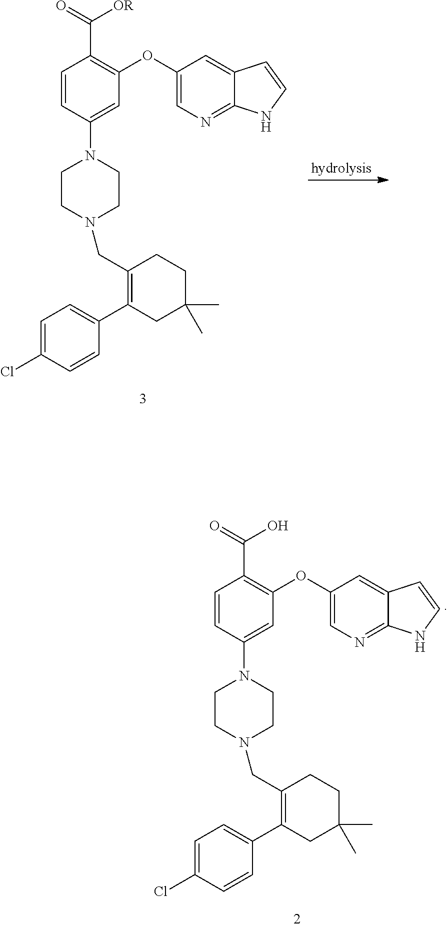

Within the context of this embodiment, examples of suitable base include, but are not limited to, sodium hydroxide, potassium hydroxide, potassium carbonate, sodium carbonate, ammonium hydroxide, potassium phosphate, and mixtures thereof. Within the context of this embodiment, the solvent may be an ether. Examples of suitable ethers include, but are not limited to, tetrahydrofuran, diglyme, diethyl ether, diisopropyl ether, dimethyl sulfoxide, dimethyl formamide, and mixtures thereof.

Within the context of this embodiment, formula 5 and formula 3 may be pharmaceutically acceptable salts of formula 5 and formula 3, respectively.

Within the context of this embodiment, formula 3 may be converted to venetoclax or a pharmaceutically acceptable salt thereof.

In another aspect, the present invention provides a process for the preparation of formula 4. In one embodiment, formula 4 may be prepared by a process that includes the step of reacting 1-hydroxymethyl-2-(4-chlorophenyl)-4,4-dimethylcyclohex-1-ene with phosphorus tribromide in the presence of a solvent.

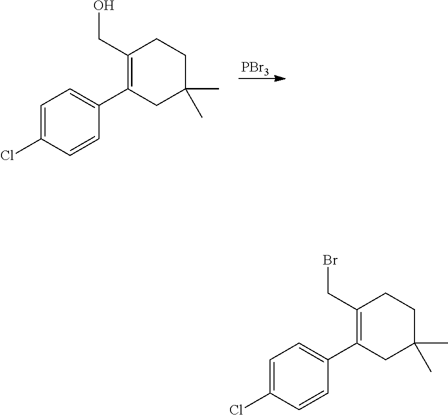

Within the context of this embodiment, the solvent may be a hydrocarbon. Examples of suitable hydrocarbons include, but are not limited to, hexane, heptane, cyclohexane, methyl cyclohexane, and mixtures thereof.

In another aspect, the present invention provides formula 5a, an acetate salt of formula 5 wherein the R moiety is a methyl group. Within the context of the invention, formula 5a may be characterized by a PXRD pattern having substantial peaks at 2Θ angles of 11.73, 13.14, 14.69, and 26.55±0.2°. Formula 5a may be further characterized by a PXRD pattern having substantial peaks at 2Θ angles of 7.49, 11.73, 12.75, 13.14, 14.69, 15.27, 16.10, 16.35, 17.27, 18.00, 18.89, 19.21, 19.86, 20.27, 21.04, 22.06, 22.36, 23.26, 23.51, 23.91, 24.36, 25.02, 25.70, 26.55, 27.30, 28.53, 29.25, 29.71, 30.49, 30.91, 31.60, 32.05, 32.89, 34.18, 34.46, 35.58, 36.28, 37.89, 38.29, 39.63, 41.32, 42.54, 43.45, 44.05, 44.92, 45.59, 48.04, 48.37, and 48.96±0.2°. The acetate salt of formula 5 may also be characterized by the PXRD pattern in FIG. 2.

In another aspect, the present invention provides a process for the preparation of an acetate salt of formula 5.

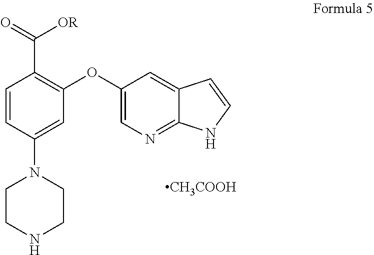

In one embodiment, an acetate salt of formula 5 may be prepared by a process that includes the steps of:
a. dissolving formula 5 in a solvent;
b. adding acetic acid; and
c. isolating an acetate salt of formula 5.

Within the context of this embodiment, this solvent may bean ether. Examples of suitable ethers include, but are not limited to, tetrahydrofuran, diethyl ether, diisopropyl ether, and mixtures thereof.

In another aspect, the present invention provides formula 3a, which is a citrate salt of formula 3 wherein the R moiety is a methyl group. Within the context of the invention, formula 3a may be characterized by a PXRD pattern having substantial peaks at 2Θ angles of 19.94, 15.88, 17.55, and 20.26±0.2°. Formula 3a may be further characterized by a PXRD pattern having substantial peaks at 2Θ angles of 6.37, 8.05, 11.52, 12.54, 13.16, 15.88, 16.43, 17.55, 19.20, 19.94, 20.26, 22.22, 22.92, 23.33, 27.02, and 30.21±0.2°. Formula 3a may be further characterized by the PXRD pattern in FIG. 1.

In another aspect, the present invention provides a process for the preparation of a citrate salt of formula 3.

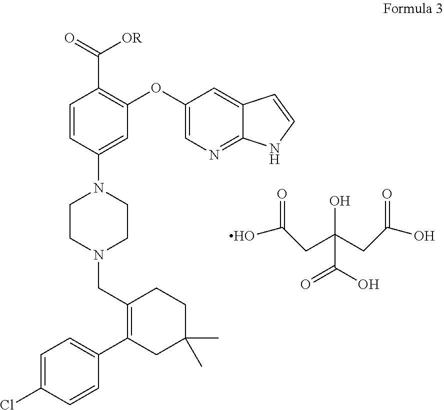

In one embodiment, a citrate salt of formula 3 may be prepared by a process that includes the steps of:
a. dissolving formula 3 and citric acid in a solvent at an elevated temperature;
b. cooling the solution; and
c. isolating a citrate salt of formula 3.

Within the context of this embodiment, the solvent may be, for example, an alcohol. Examples of suitable alcohols include, but are not limited to, methanol, ethanol, isopropanol, and mixtures thereof.

In another aspect, the present invention provides a process for the preparation of venetoclax. In one embodiment, venetoclax may be prepared by a process that includes the steps of:
a. dissolving venetoclax in a solvent to form a solution;
b. cooling the solution; and
c. isolating amorphous venetoclax.

In some embodiments, the dissolving step is carried out at an elevated temperature. In some embodiments, the cooling step is carried out at a temperature of about 0° C. to about 15° C. Within the context of this embodiment, examples of suitable solvents include, but are not limited to, acetonitrile, acetone, methyl isobutyl ketone, and mixtures thereof.

### BRIEF DESCRIPTION OF THE DRAWINGS

Further aspects of the present disclosure together with additional features contributing thereto and advantages accruing there from will be apparent from the following description of embodiments of the disclosure which are shown in the accompanying drawings wherein:

FIG. 1 is an X-ray powder diffraction pattern of crystalline citrate salt of formula 3a,

FIG. 2 is an X-ray powder diffraction pattern of crystalline acetate salt of formula 5a; and

FIG. 3 is an X-ray powder diffraction pattern of amorphous venetoclax.

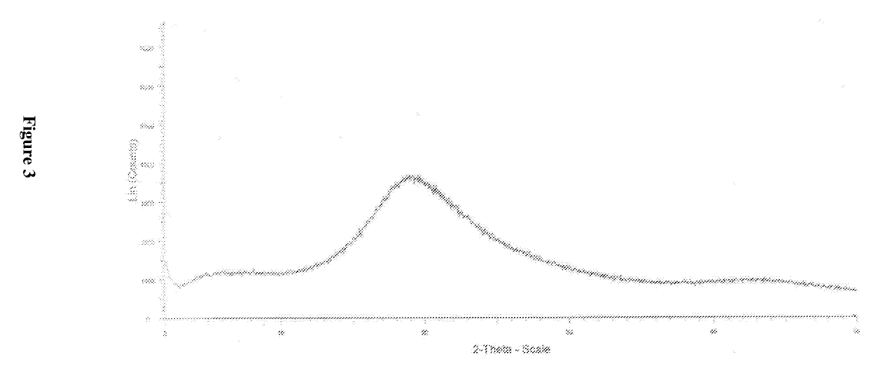

### DETAILED DESCRIPTION OF THE INVENTION

The compounds disclosed herein may be characterized by powder X-ray diffraction (PXRD). Thus, samples of compounds prepared by methods disclosed herein were analyzed on a BRUKER D-8 Discover powder diffractometer equipped with goniometer of θ/2θ configuration and Lynx Eye detector. The Cu-anode X-ray tube was operated at 40 kV and 30 mA. The experiments were conducted over the 2θ range of 2.0°-50.0°, 0.030° step size and 0.4 seconds step time.

The present invention provides novel synthetic schemes for the synthesis of venetoclax. Within the context of the present invention, novel intermediates are generated that may be useful for preparing venetoclax. Together, these schemes and intermediates provide an improved, efficient method for the synthesis of venetoclax.

In one aspect, the present invention provides a process for the preparation of venetoclax, shown as Formula-I below.

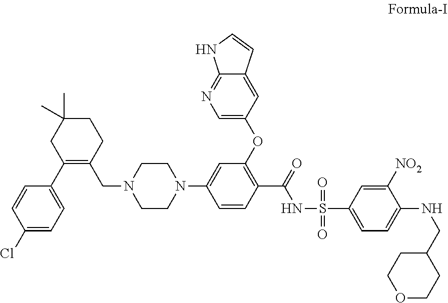 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In one embodiment, venetoclax may be prepared by a process that includes the following steps:
a) reacting formula 8 with formula 7 in the presence of a base to get formula 6;

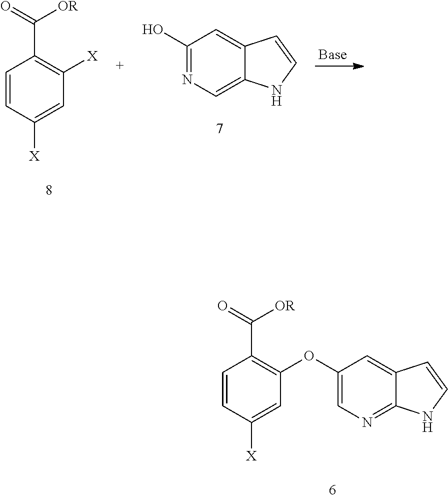

b) treating formula 6 with piperazine to get formula 5;

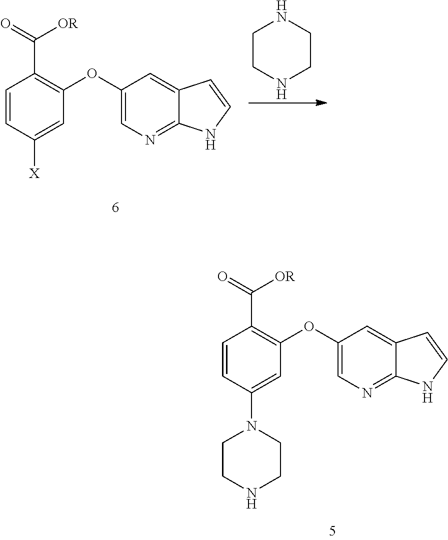

c) condensing formula 5 with formula 4 in the presence of a base to obtain formula 3; and

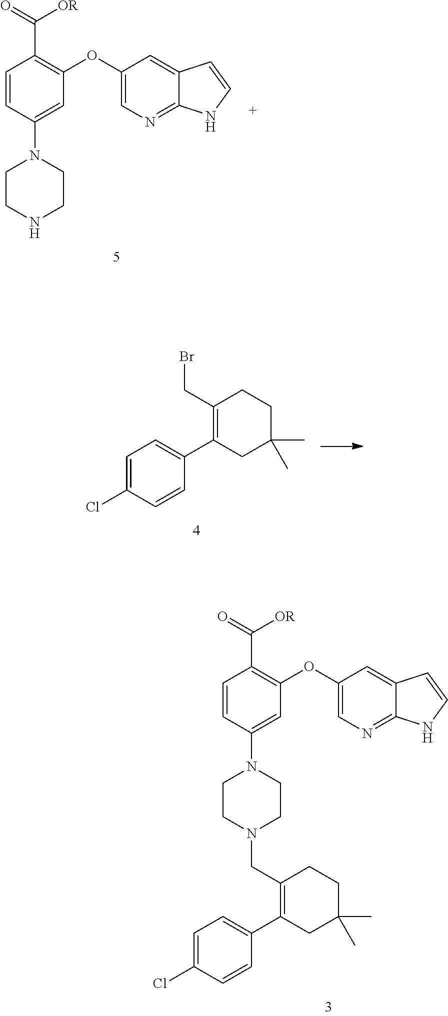

d) hydrolyzing formula 3 to obtain formula 2; and

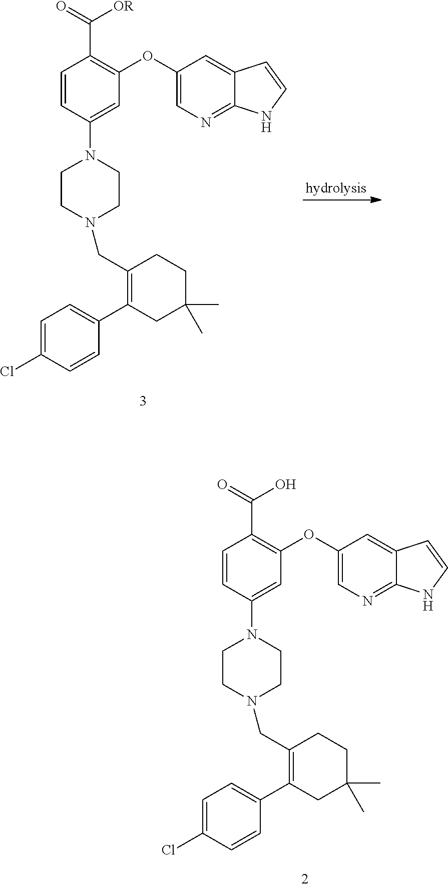

e) reacting formula 2 with formula 9 to obtain venetoclax of Formula-I.

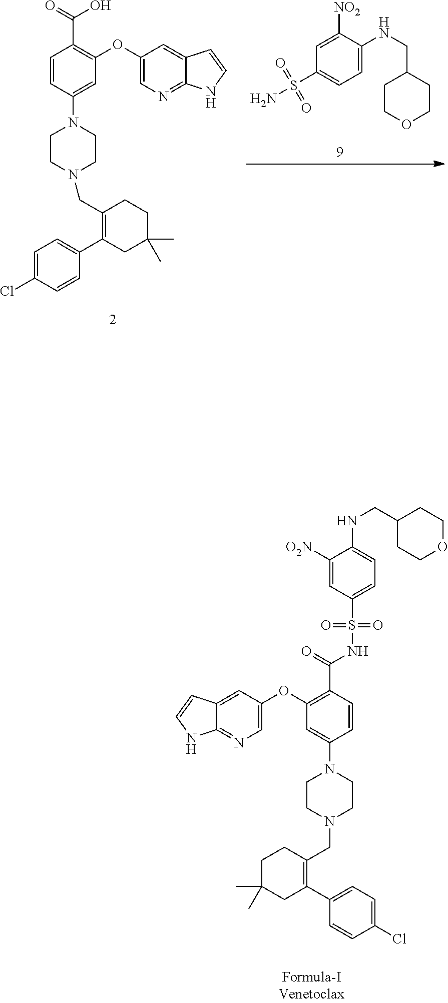 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Within the context of this embodiment, R is a C₁₋₄ alkyl and X is a halogen, for example, fluorine, chlorine, bromine, or iodine.

According to the present embodiment, formula 8 may be reacted with formula 7 in the presence of a base to get formula 6. One of skill in the art will be familiar with bases that may be used. For example, the base may be, but is not limited to, sodium hydroxide, potassium hydroxide, ammonium hydroxide, potassium phosphate, or mixtures thereof. In some embodiments, this reaction is carried out in the presence of potassium phosphate. This reaction may be carried out in a suitable solvent. One of skill in the art will be familiar with suitable solvents. Examples of suitable solvents include, but are not limited to, ether solvents, dimethylformamide, toluene, 2-methyl-tetrahydrofuran, tetrahydrofuran, and mixtures thereof. Examples of suitable ethers include, but are not limited to, tetrahydrofuran, diglyme, diethyl ether, diisopropyl ether, and mixtures thereof. In some embodiments, this reaction is carried out in diglyme.

Formula 6 may then be reacted with piperazine to get formula 5. One of skill in the art will be familiar with a solvent in which this reaction may be performed. For example, the solvent may be, but is not limited to, dimethyl sulfoxide, dimethyl formamide, or mixtures thereof. In some embodiments, this reaction is carried out in dimethyl sulfoxide.

Next, formula 5 may be reacted with formula 4 in the presence of a base to get formula 3. One of skill in the art will be familiar with a suitable base. For example, suitable bases include, but are not limited to, triethylamine, pyridine, diisopropylethylamine, sodium carbonate, potassium carbonate, sodium hydroxide, potassium hydroxide, ammonium hydroxide, potassium phosphate, and mixtures thereof. In some embodiments, this reaction is carried out in potassium carbonate. One of skill in the art will be familiar with a solvent that may be used to carry out this reaction. Examples of useful solvents include, but are not limited to, tetrahydrofuran, 2-methyl-tetrahydrofuran, N-methyl-2-pyrrolidone, ethyl acetate, dimethyl sulfoxide, dimethyl formamide, or mixtures thereof.

Formula 3 may then be hydrolyzed to get formula 2. One of skill in the art will be familiar with methods for hydrolyzing formula 3, for example, by treating formula 3 with a base. Suitable bases include, but are not limited to, sodium hydroxide, potassium hydroxide, ammonium hydroxide, potassium phosphate, and mixtures thereof. In some embodiments, this reaction is carried out in sodium hydroxide. One of skill in the art will be familiar with a solvent in which this reaction may be performed. For example, suitable solvents include, but are not limited to, alcohols, dichloromethane, dimethyl sulfoxide, dimethyl formamide, 2-methyltetrahydrofuran, tetrahydrofuran, ethyl acetate, N-methyl-2-pyrrolidone, water, and mixtures thereof. Examples of useful alcohols include, but are not limited to, methanol, ethanol, isopropanol, and mixtures thereof. In some embodiments, hydrolysis with a base is carried out in dimethyl formamide.

Formula 2 may then be reacted with formula 9 in the presence of a suitable reagent to obtain venetoclax. This may be carried out in a suitable solvent, for example, a chlorinated solvent. One of skill in the art will be familiar with suitable chlorinate solvents that would be useful for reacting formula 2 with formula 9. In some embodiments, dichloromethane is used. Within the context of this embodiment, the suitable reagent may be a coupling agent. One of skill in the art will be familiar with a variety of different reagents that may be used to couple formula 2 and formula 9, for example (but not limited to), N-(3-dimethylaminopropyl)-N′-ethylcarbodiimide (EDC), 4-(dimethylamino)pyridine (DMAP), 2-(benzoylcarbamothioylamino)-5,5-dimethyl-4,7-dihydrothieno[2,3-c]pyran-3-carboxylic acid (CID), (benzotriazol-1-yloxy)tripyrrolidinophosphonium hexafluorophosphate (PY-BOP), or 1-[bis(dimethylamino)methylene]-1H-1,2,3-triazolo[4,5-b]pyridinium 3-oxide hexafluorophosphate (HATU).

Within the context of this invention, a salt of any one of formulas 5, 3, or I (venetoclax) may be used or prepared in lieu of the free base form.

Methods for converting compounds into their acid salt forms are well known in the art, and may be carried out, for example, by reacting a free base moiety on the compound with a suitable reagent.

Examples of suitable acids include, for example, inorganic acids or organic acids. Examples of suitable inorganic acids include hydrochloric acid, hydrobromic acid, phosphoric acid, sulfuric acid, and perchloric acid. Suitable organic acids include, for example, acetic acid, oxalic acid, maleic acid, tartaric acid, citric acid, succinic acid, and malonic acid. A pharmaceutically acceptable salt may alternatively be prepared by other methods well known in the art, for example, ion exchange. Additional examples of suitable salts include, for example, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, (R,S)-malate, (S)-malate, maleate, malonate, methanesulfonate, 2-naphthalenesulfonate, nicotinate, nitrate, oleate, oxalate, palmitate, pamoate, pectinate, persulfate, 3-phenylpropionate, phosphate, phthalate, picrate, pivalate, propionate, stearate, succinate, sulfate, tartrate, thiocyanate, p-toluenesulfonate, undecanoate, and valerate salts.

In a particularly useful embodiment, the citrate salt of formula 3 is used.

Thus, in one aspect, the present invention provides a process for preparing a citrate salt of formula 3. In one embodiment, a citrate salt of formula 3 may be prepared by a process that includes the following steps:
a) dissolving formula 3 and citric acid in a solvent to form a solution;
b) cooling the solution; and
c) isolating a citrate salt of formula 3.

According to the present embodiment, formula 3 and citric acid may be dissolved in a solvent. Within the context of this embodiment, the “R” group on formula 3 is a C₁₋₄ alkyl. One of skill in the art will be familiar with suitable solvents. For example, an alcohol solvent may be used. Examples of suitable alcohol solvents include, but are not limited to, methanol, ethanol, isopropanol, and mixture thereof. An elevated temperature may be used to facilitate dissolution of formula 3 and citric acid. For example, in some embodiments, a temperature of about 50° C. to about 65° C. is used.

Within the context of the invention, the term “about” when modifying an absolute measurement, such as time, mass, or volume, is meant to mean the recited value plus or minus 10% of that value (e.g., in certain embodiments, “about” includes plus or minus 5%, or plus or minus 2%, or plus or minus 1% of that value). Within the context of the invention, the term “about” when modifying a temperature measurement is meant to mean the recited temperature plus or minus five degrees.

Next, the solution may be cooled, for example, to room temperature. In some embodiments, this cooling facilitates crystallization of the citrate salt of formula 3, which may be isolated by methods well known in the art. For example, in some embodiments, isolation is carried out by filtering the reaction mixture and collecting a solid.

In another particularly useful embodiment, an acetate salt of formula 5 is used.

Thus the present invention provides a process for preparing an acetate salt of formula 5. In one embodiment, crystalline acetate salt of formula 5 may be prepared by a process that includes the following steps:
a) dissolving formula 5 in a solvent;
b) adding acetic acid; and
c) isolating a crystalline acetate salt formula 5.

According to the present embodiment, formula 5 may be dissolved in a solvent. Within the context of this embodiment, the “R” group on formula 5 is a C₁-C₄ alkyl. In particularly useful embodiments, R is methyl. One of skill in the art will be familiar with solvent that may be used, for example, ether solvents. Examples of suitable ether solvents include, but are not limited to, tetrahydrofuran, diethyl ether, diisopropyl ether, and mixture thereof. In some embodiments, formula 5 is dissolved in tetrahydrofuran.

Next, acetic acid may be added and the acetate salt of formula 5 may be isolated. In some embodiments, cooling of the solution after addition of acetic acid is used to facilitate formation of an acetate salt of formula 5. For example, the solution may be cooled to room temperature. Isolation may be carried out by methods well known in the art, for example, by crystallization

In one embodiment, Scheme-I below depicts one embodiment of the process described above for the preparation of venetoclax:

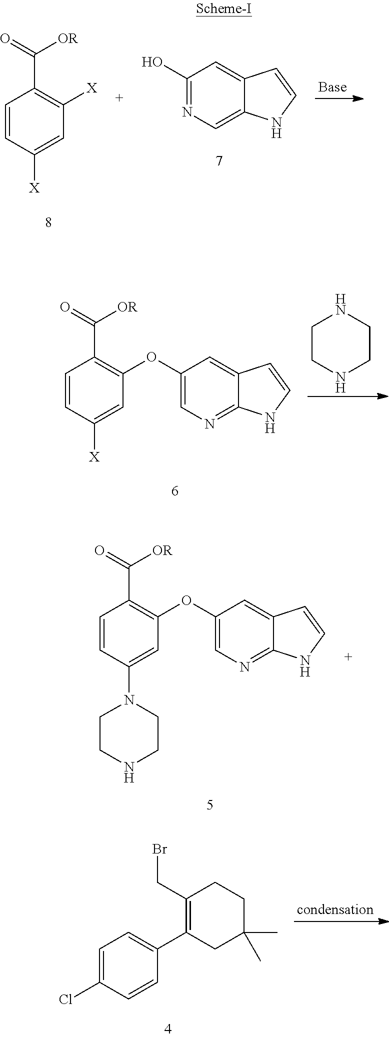

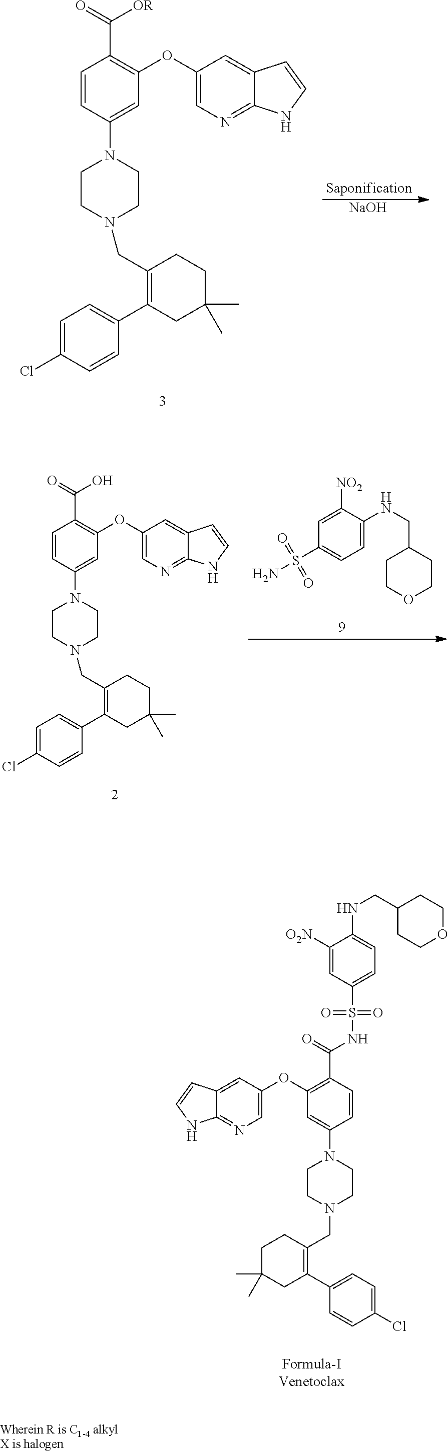

In another aspect, the present invention provides processes for the preparation of intermediates used in the above-disclosed process for preparing venetoclax as represented in schemes i-iv below.

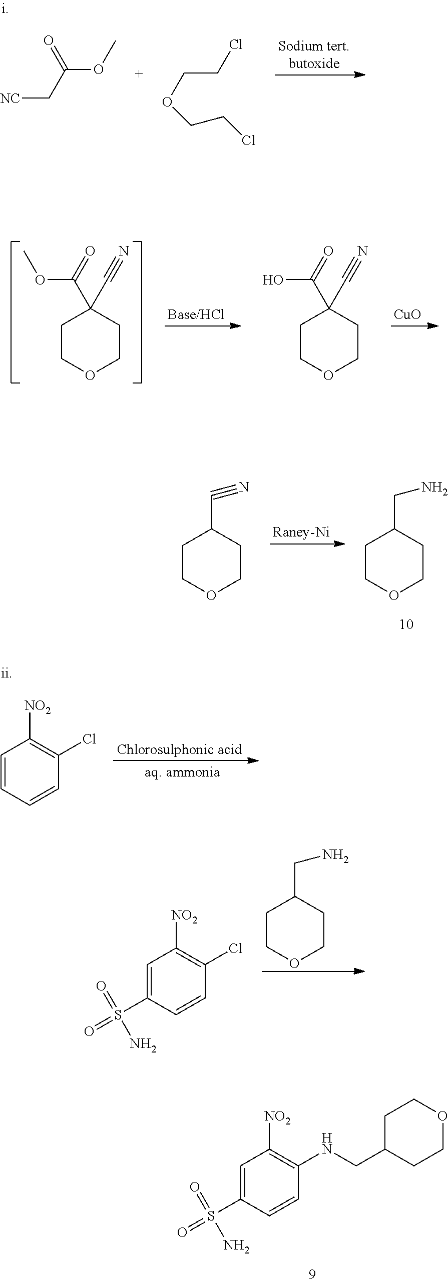

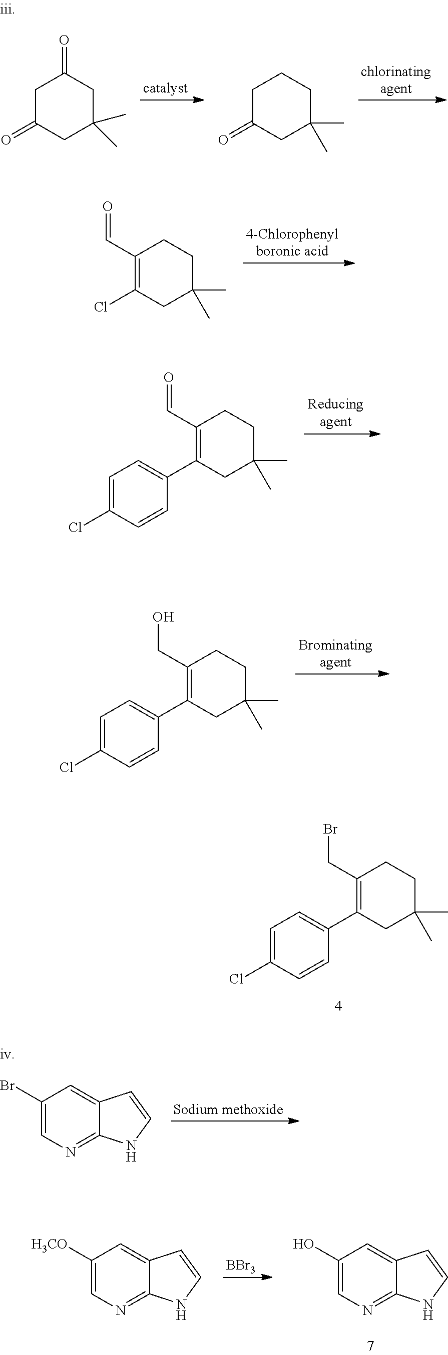

In another aspect, the present invention provides a particular embodiment of citrate salt of formula 3 wherein R is methyl. This embodiment is shown below as Formula 3a.

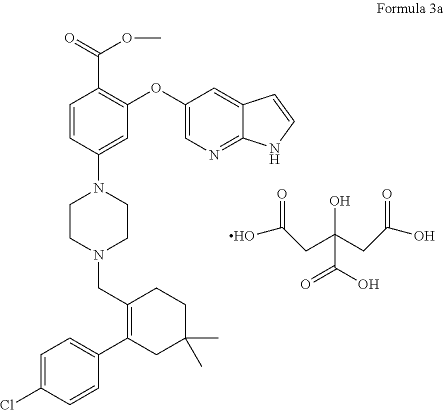

Within the context of the invention, formula 3a may be characterized by a PXRD pattern having substantial peaks at 2Θ angles of 15.88, 17.55, 19.94, and 20.26±0.2°.

Formula 3a may be further characterized by a PXRD pattern having substantial peaks at 2Θ angles of 6.37, 8.05, 11.52, 12.54, 13.16, 15.88, 16.43, 17.55, 19.20, 19.94, 20.26, 22.22, 22.92, 23.33, 27.02, and 30.21±0.2°.

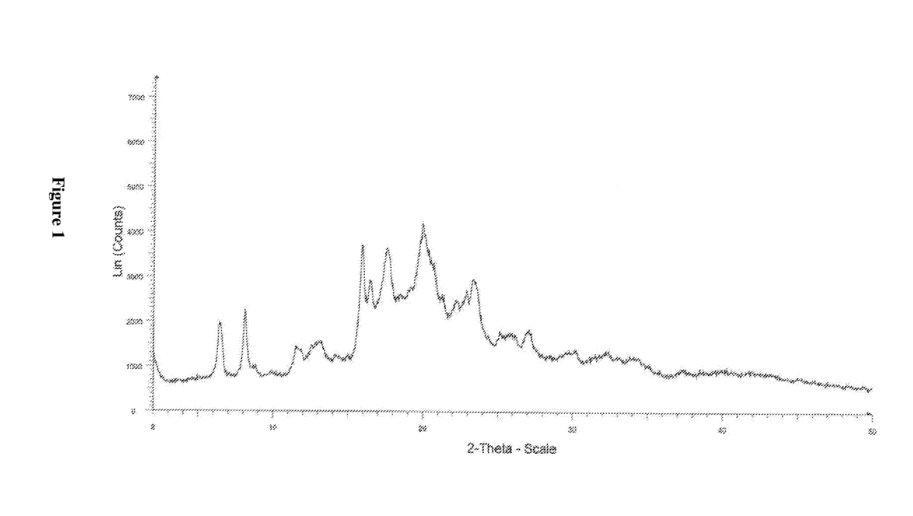

Formula 3a may further be characterized by the PXRD pattern as shown in FIG. 1.

In another aspect, the present invention provides a particular embodiment of an acetate salt of formula 5 wherein R is methyl. This embodiment is shown below as formula 5a.

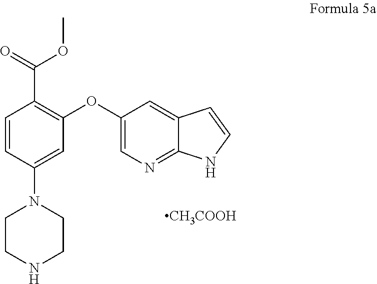

Within the context of the invention, formula 5a may be characterized by a PXRD pattern having substantial peaks at 2Θ angles of 11.73, 13.14, 14.69, and 26.55±0.2°.

Formula 5a may be further characterized by a PXRD having substantial peaks at 2Θ angles of 7.49, 11.73, 13.14, 14.69, 26.55±0.2°.

Formula 5a may be further characterized by a PXRD having substantial peaks at 2Θ angles of 7.49, 11.73, 12.75, 13.14, 14.69, 15.27, 16.10, 16.35, 17.27, 18.00, 18.89, 19.21, 19.86, 20.27, 21.04, 22.06, 22.36, 23.26, 23.51, 23.91, 24.36, 25.02, 25.70, 26.55, 27.30, 28.53, 29.25, 29.71, 30.49, 30.91, 31.60, 32.05, 32.89, 34.18, 34.46, 35.58, 36.28, 37.89, 38.29, 39.63, 41.32, 42.54, 43.45, 44.05, 44.92, 45.59, 48.04, 48.37, and 48.96±0.2°.

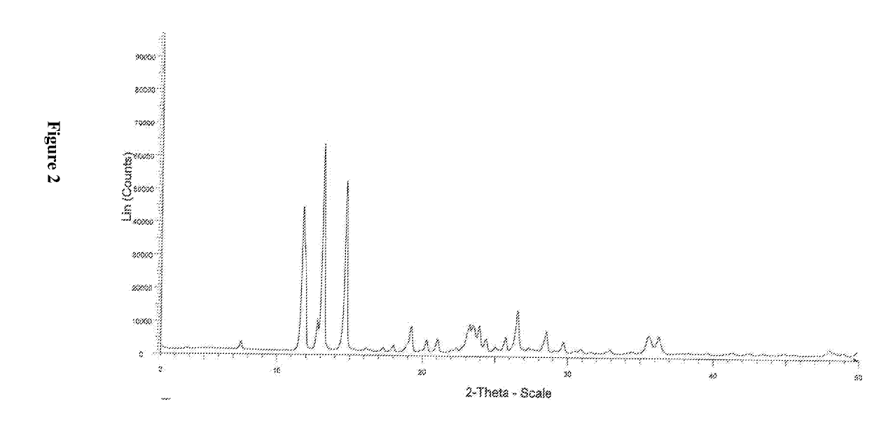

Formula 5a may also be characterized by the PXRD pattern in FIG. 2.

In another aspect, the present invention provides formula 4, shown below:

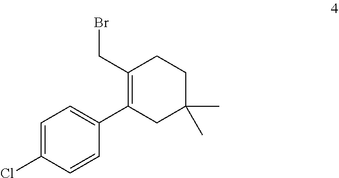 [SMILES: CC1(C)CCC(CBr)=C(c2ccc(Cl)cc2)C1]

In another aspect, the present invention provides a process for the preparation of formula 4. In one embodiment, formula 4 may be prepared by reacting 1-hydroxymethyl-2-(4-chlorophenyl)-4,4-dimethylcyclohex-1-ene with phosphorus tribromide, as shown below.

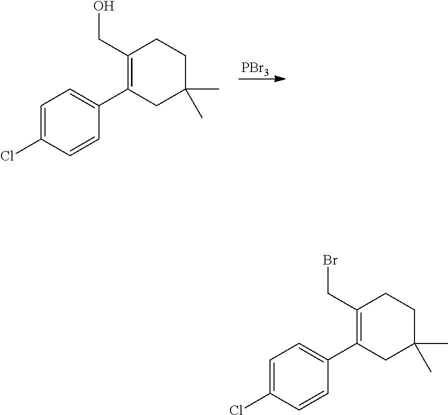

This reaction may be carried out in the presence of a base in a suitable solvent. Examples of suitable bases include, but are not limited to, pyridine, triethylamine, and diisopropylethylamine. Examples of suitable solvents include, but are not limited to, dichloromethane, hydrocarbons, and mixtures thereof. Examples of suitable hydrocarbons include, but are not limited to, hexane, heptane, cyclohexane, methylcyclohexane, and mixture thereof.

In another aspect, the present invention provides a process for the preparation of amorphous venetoclax.

In one embodiment, amorphous venetoclax may be prepared by a process that includes the following steps:
a) dissolving venetoclax in an organic solvent at elevated temperature;
b) slowly cooling the solution to 0° C. to 15° C.; and
c) isolating amorphous venetoclax.

According to the present embodiment, venetoclax may be dissolved in a solvent at elevated temperature. One of skill in the art will be familiar with solvents that may be used for dissolving venetoclax. Examples of suitable solvents include, but are not limited to, alcohols, ethers, ketones, acetonitrile, and mixtures thereof. Examples of suitable alcohols include, but are not limited to, methanol, ethanol, isopropanol, and mixtures thereof. Suitable ketones include, but are not limited to acetone, methyl isobutyl ketone, and mixtures thereof. Suitable ethers include, but are not limited to tetrahydrofuran, diglyme, diethyl ether, diisopropyl ether, and mixtures thereof. Dissolution may be carried out at an elevated temperature. In some embodiments, a temperature of about 50° C. to a bout 65° C. is used.

Next, the solution may be slowly cooled to facilitate formation of a precipitate. In particularly useful embodiments, a temperature of about 5° C. to about 15° C. is used. Within the context of this embodiment, the cooling step may be carried out slowly by placing the reaction vessel in an ice bath.

Amorphous venetoclax may then be isolated by methods well known in the art. For example, in some embodiments, the solution is filtered to get amorphous venetoclax

Another aspect of the present invention provides yet another process for the preparation of venetoclax, illustrated below in Schemes-II to -XIII, and Schemes-XI to -XIII. Several intermediate depicted in these aforementioned schemes are disclosed in Scheme-X, Scheme-XIV, and Scheme-XV. Within the context of each of these schemes, X and X₁ are halogen, R is a C₁-C₄ alkyl group, P is a hydroxy protecting group, and G is —H or an amine protecting group. Within the context of the invention the alkyl group may be straight or branched.

The terms “amine protecting group” as well as “hydroxyl protecting group” are well known and understood in the art. Examples of suitable amine protecting groups, suitable hydroxyl protecting groups, as well as suitable conditions for protecting and deprotecting, can be found in prior art, such as J. F. W. McOmie, “Protective Groups in Organic Chemistry”, Plenum Press, London and New York 1973; T. W. Greene and P. G. M. Wuts, “Protective Groups in Organic Synthesis”, Third edition, Wiley, New York 1999; “The Peptides”; Volume 3 (editors: E. Gross and J. Meienhofer), Academic Press, London and New York 1981; in “Methoden der organischen Chemie”, Houben-Weyl, 4th edition, Vol. 15/1, Georg Thieme Verlag, Stuttgart 1974; H.-D. Jakubke and H. Jescheit, “Aminosauren, Peptide, Proteine”, Verlag Chemie, Weinheim, Deerfield Beach, and Basel 1982; and Jochen Lehmann, “Chemie der Kohlenhydrate: Monosaccharide and Derivate”, Georg Thieme Verlag, Stuttgart 1974.

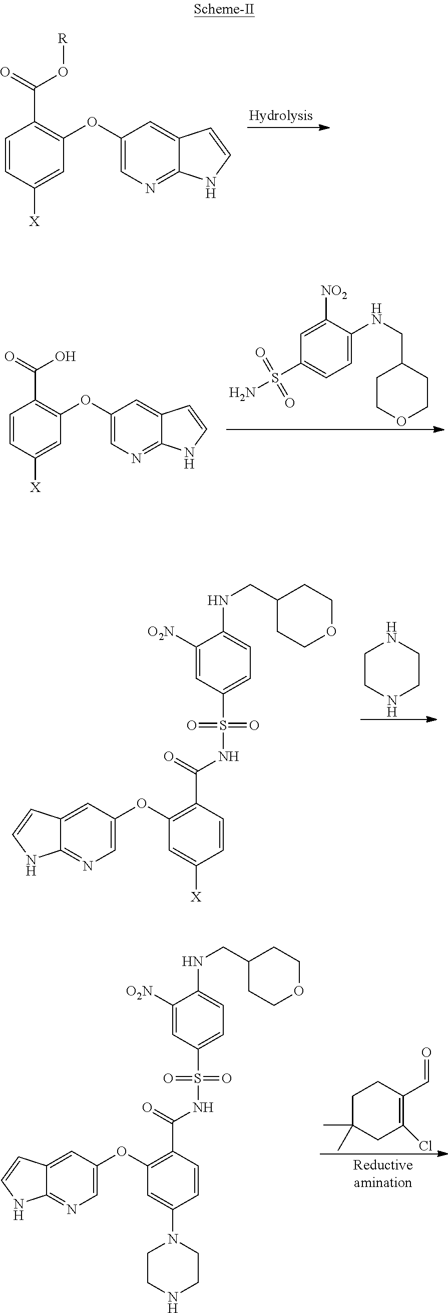

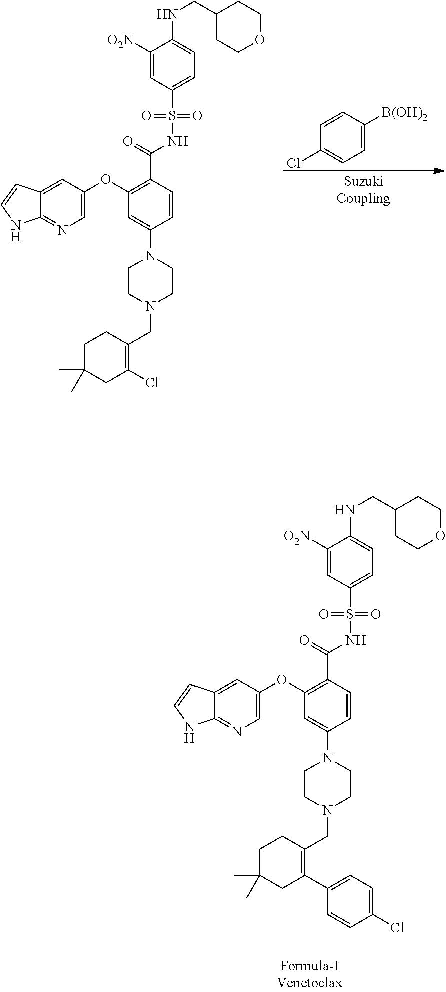 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(Cl)C1 ; OB(O)c1ccc(Cl)cc1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

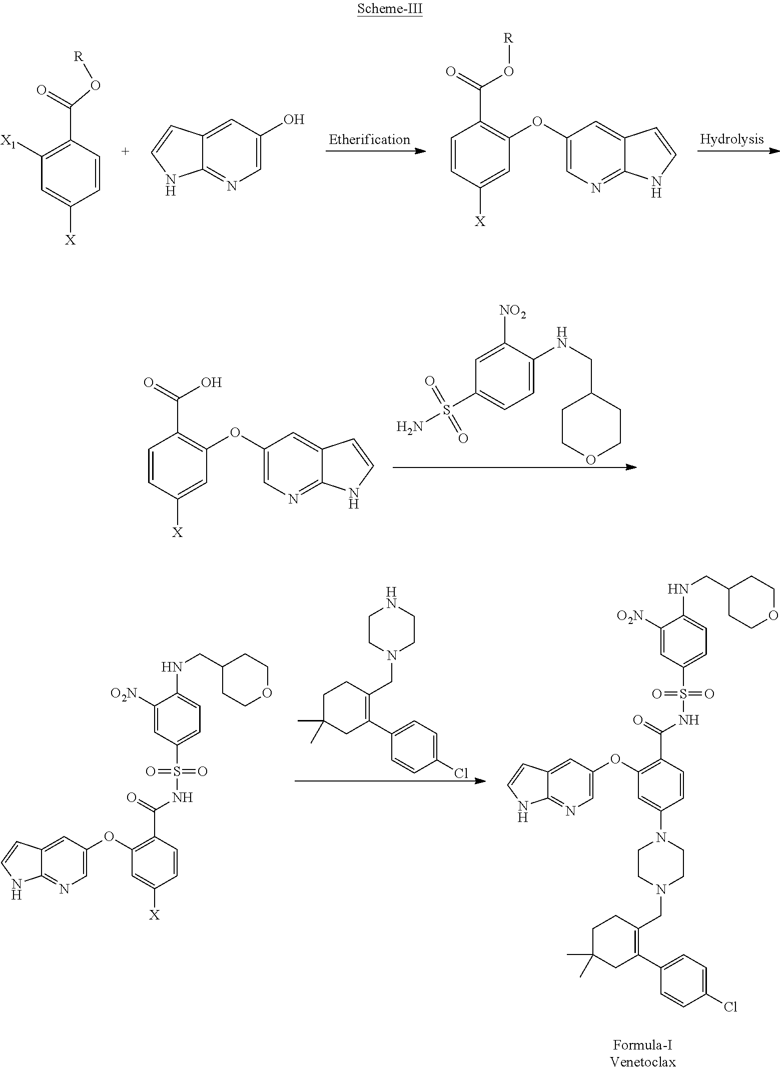

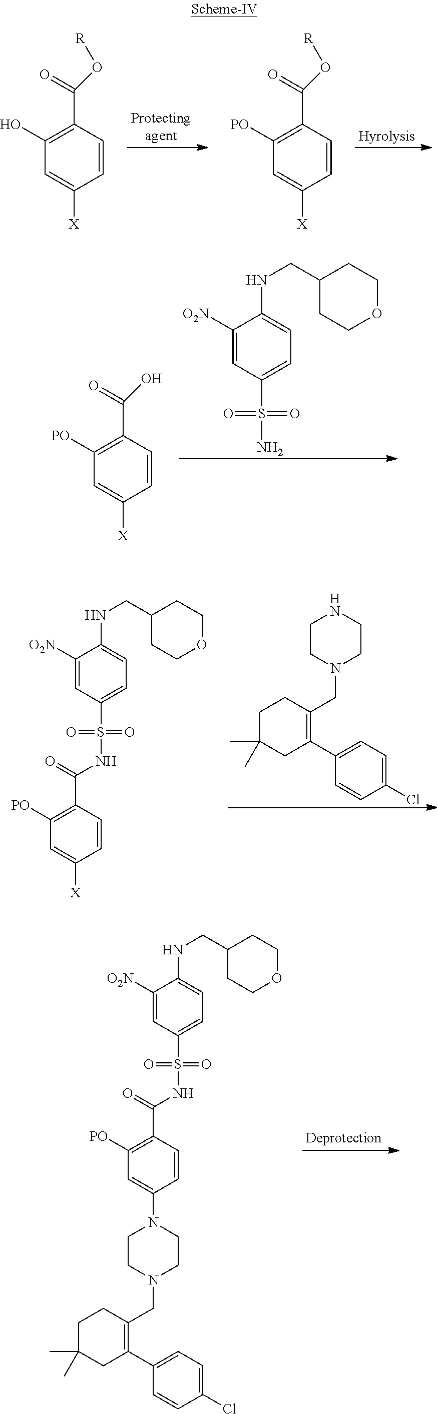

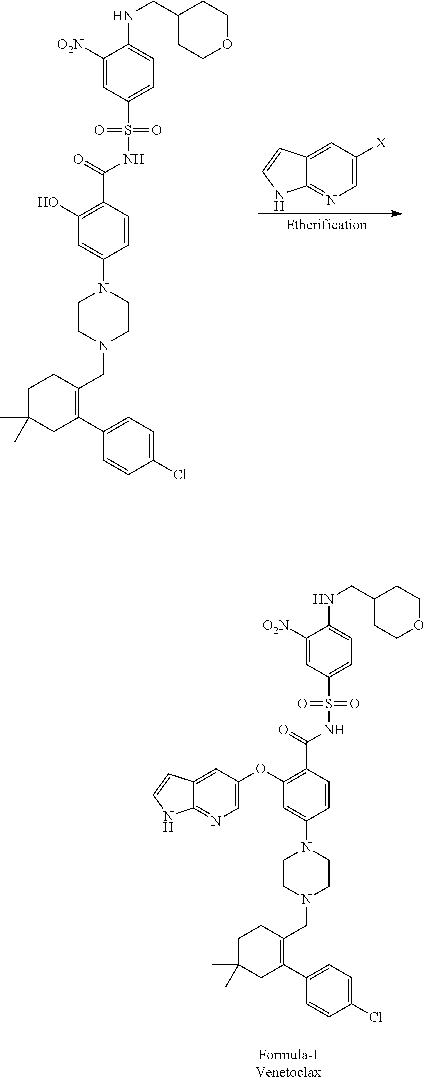

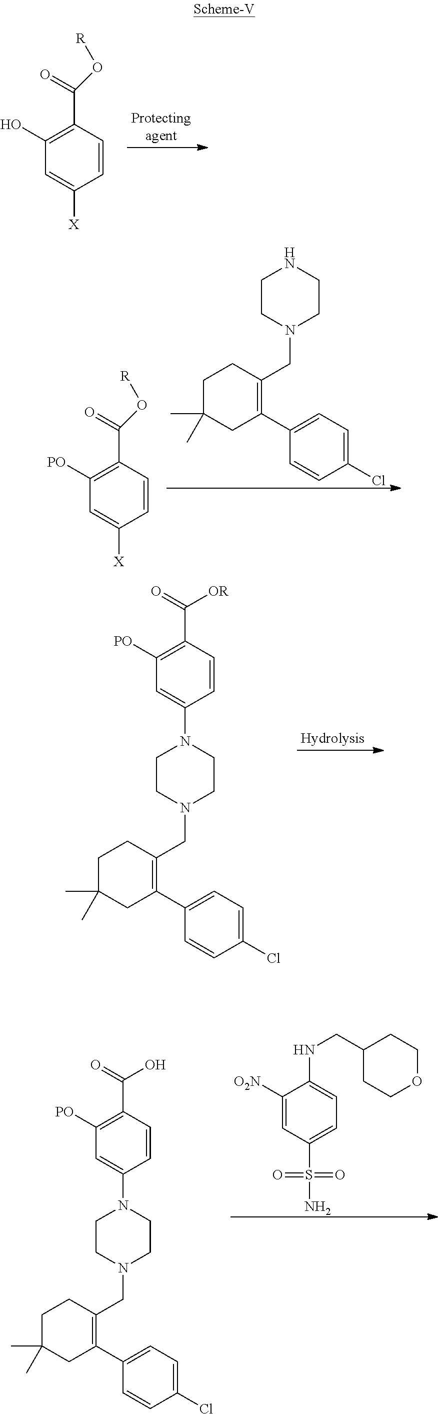

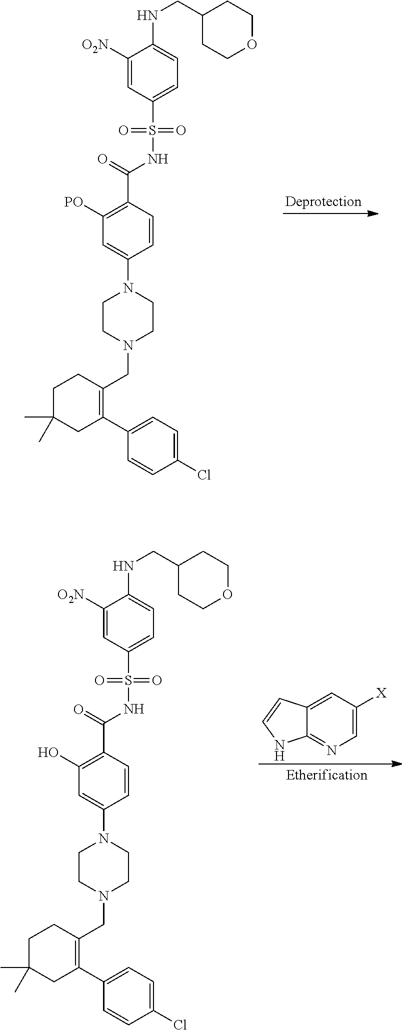

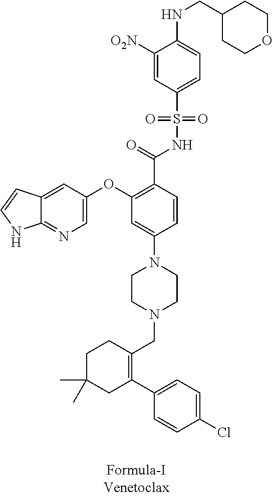 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

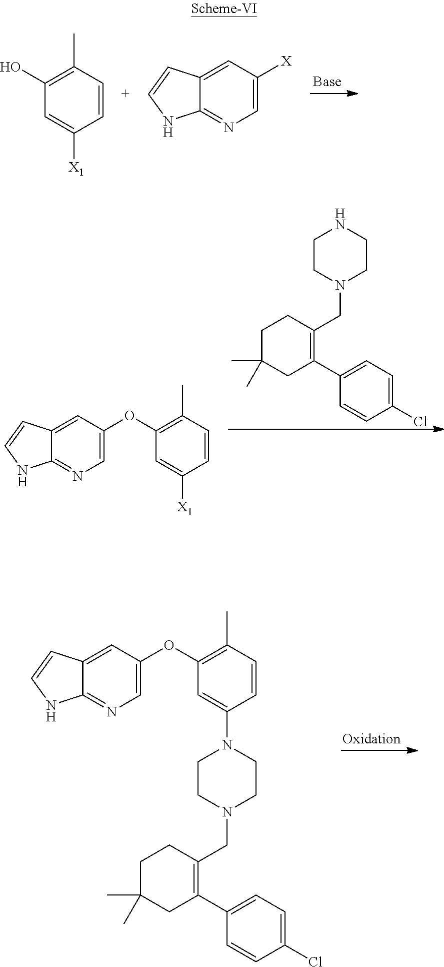

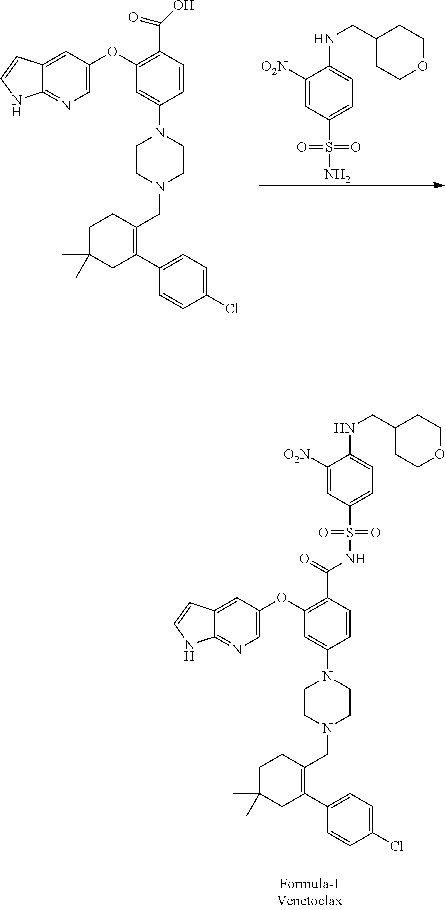 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

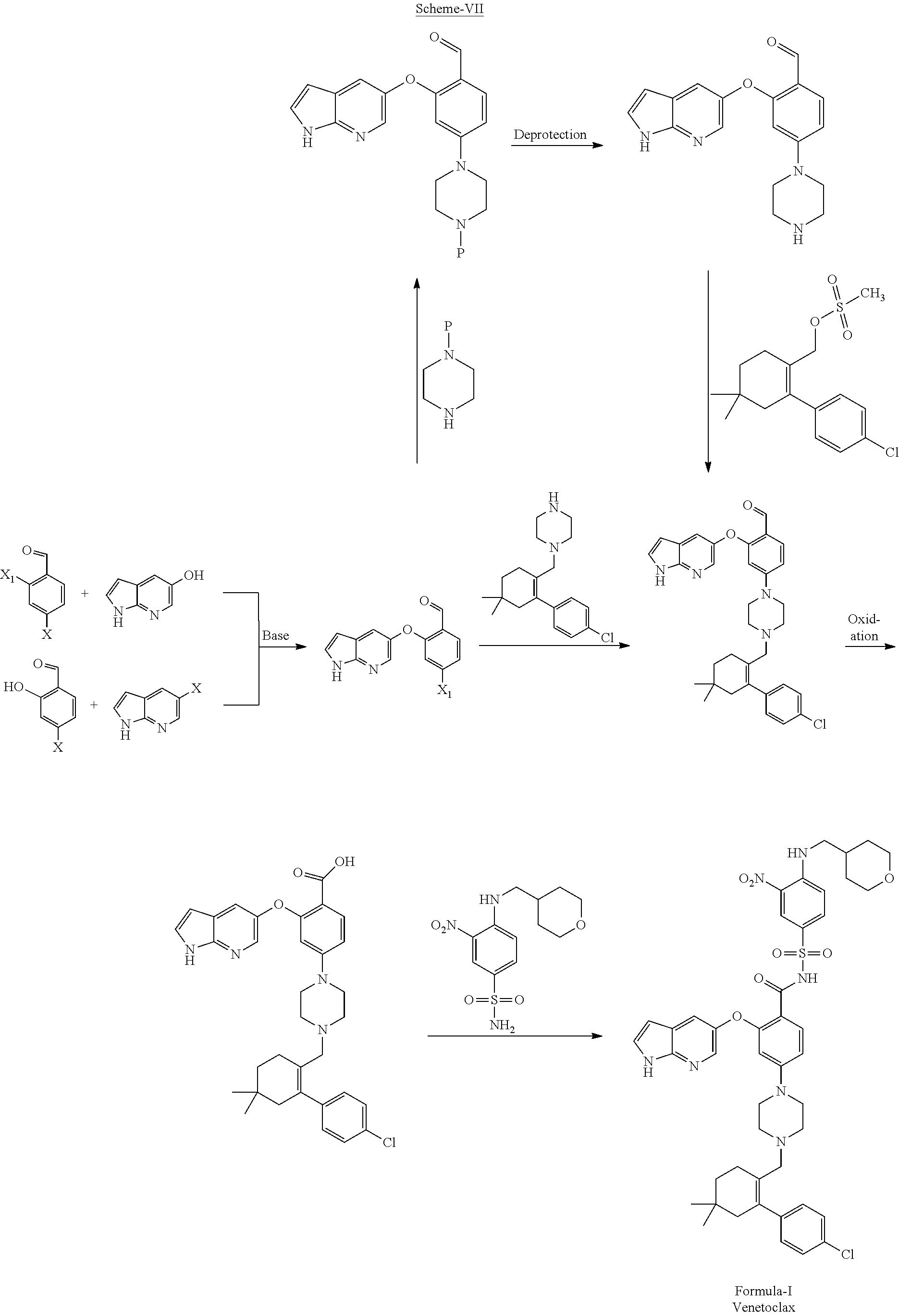

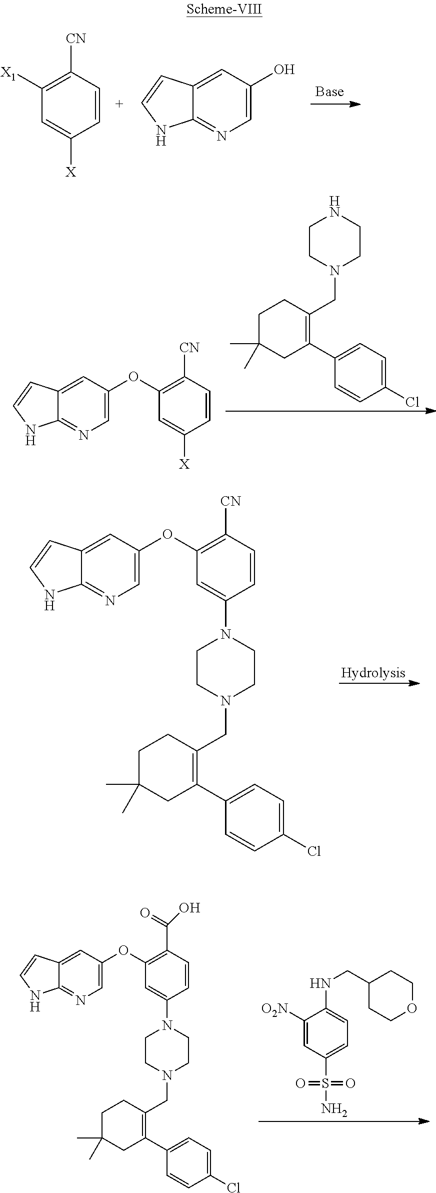

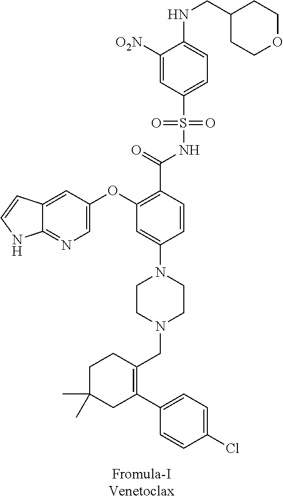 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

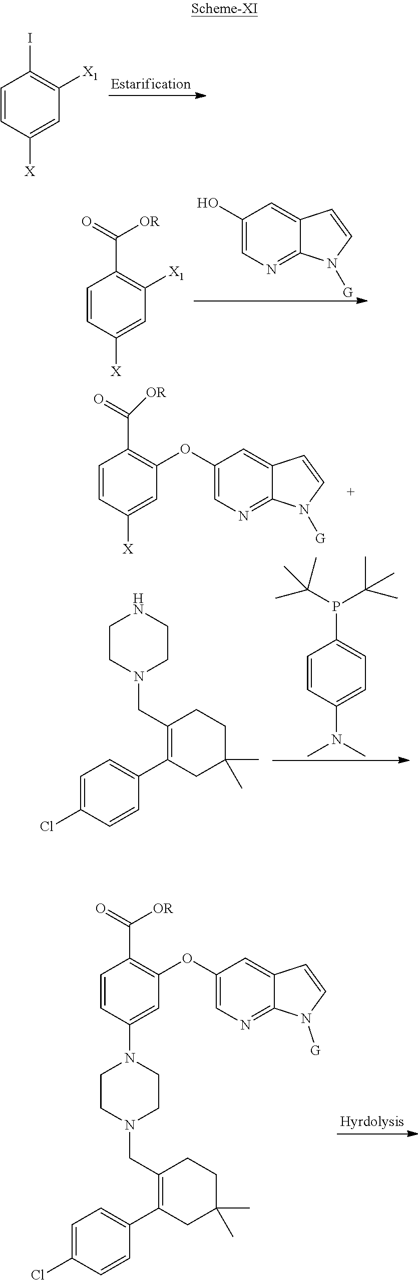

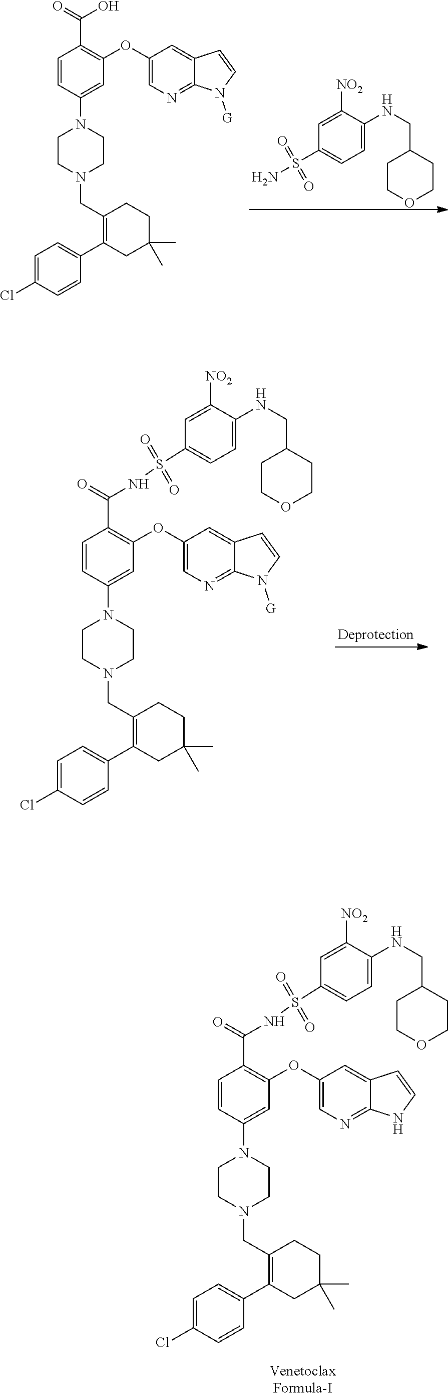

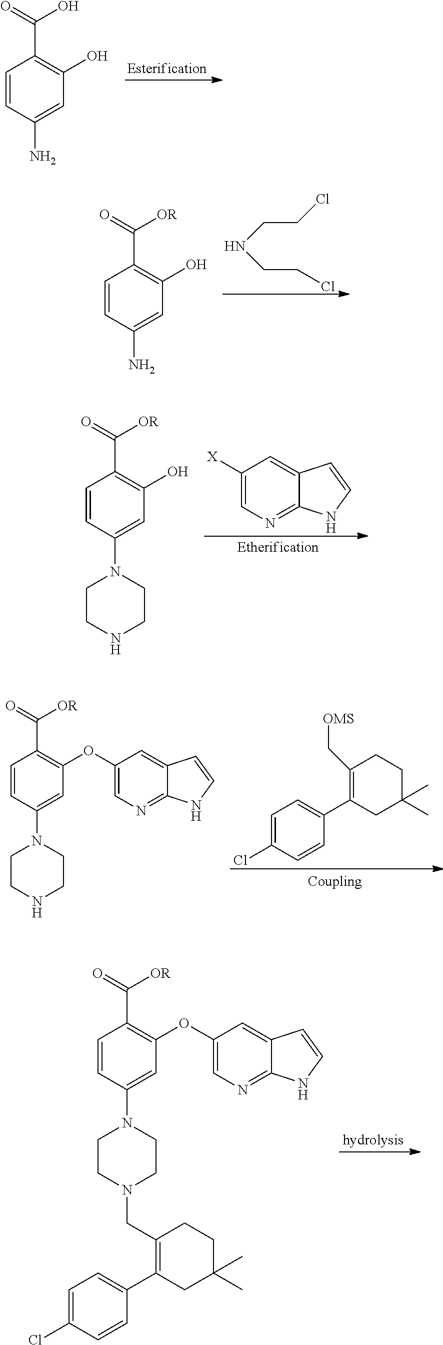

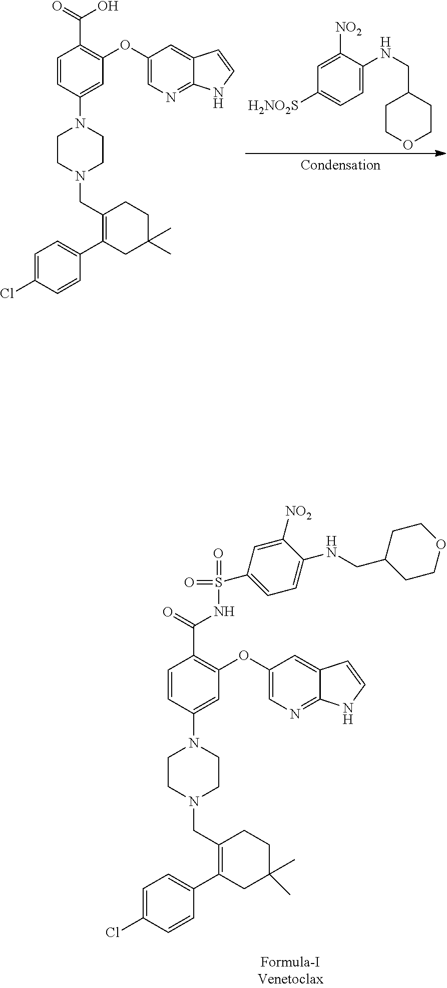 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

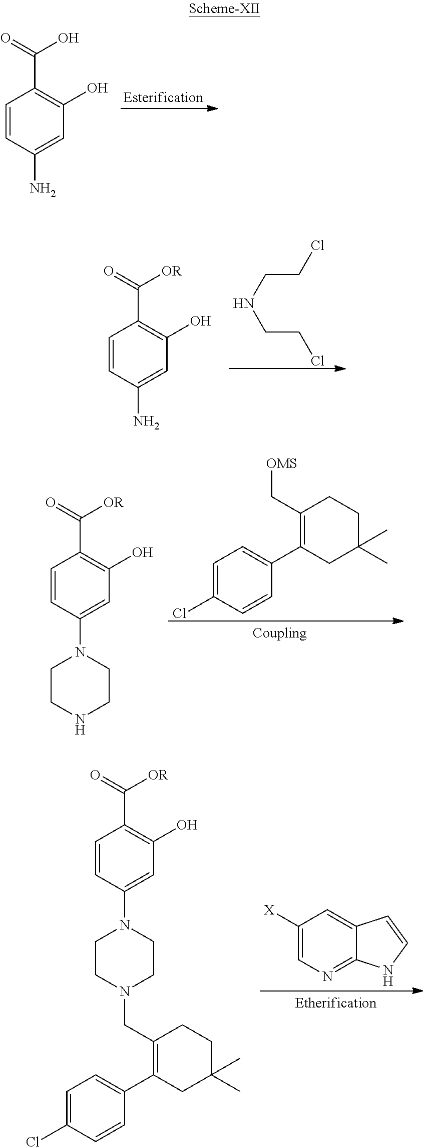

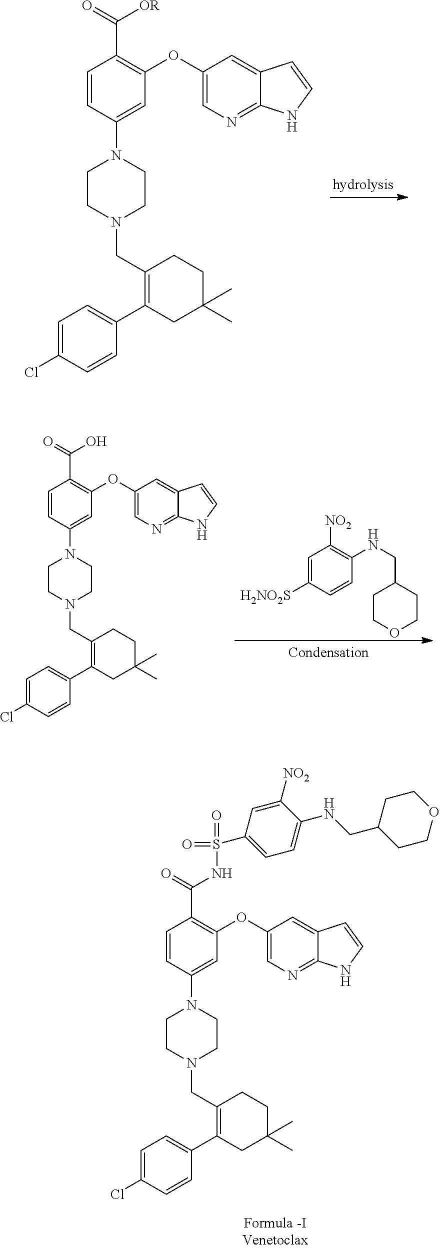

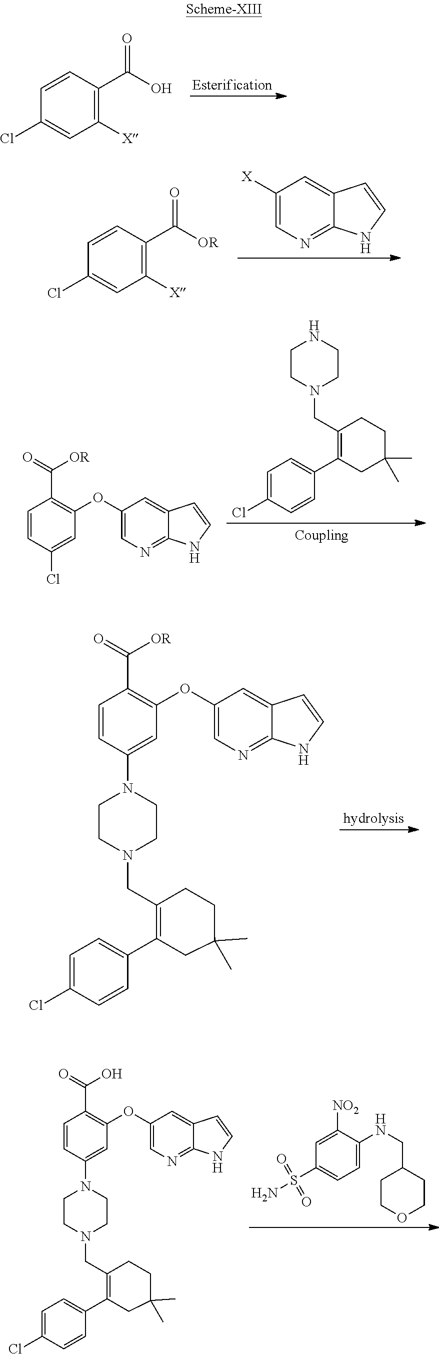

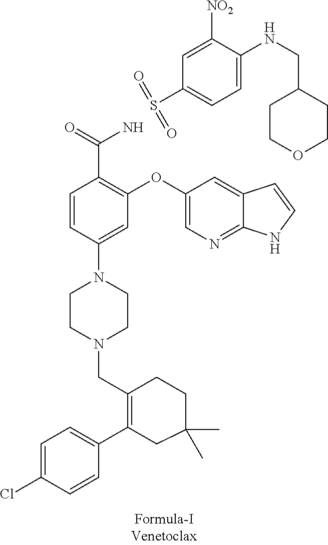

Within the context of the invention, the mesylate-protected (2-(4-chlorophenyl)-4,4-dimethylcyclohex-1-enyl)methanol depicted in Schemes-XI, —XII, and —XIII may be prepared according to the process depicted below in Scheme-XIV.

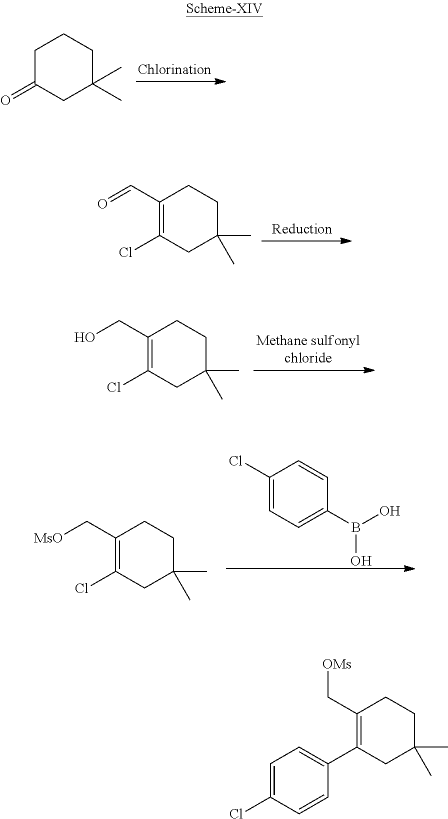

In another embodiment, formula 6, where R═H, may be prepared by the steps depicted below in Scheme-XV.

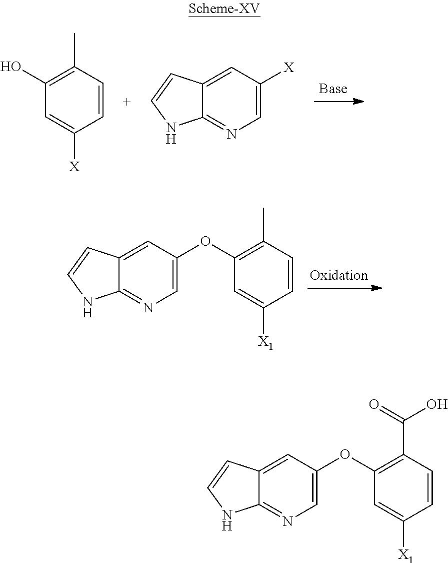

Venetoclax as well as pharmaceutically acceptable salts thereof, prepared by methods disclosed herein, may be used to formulate an oral dosage form, for example, a tablet or a capsule. When administered to patients, the venetoclax and pharmaceutically acceptable salts thereof of the present invention may be useful in therapy for the treatment of chronic lymphocytic leukemia.

Venetoclax or pharmaceutically acceptable salts thereof, prepared by methods disclosed herein, may be formulated into a tablet which may contain additional inactive ingredients such as copovidone, colloidal silicon dioxide, polysorbate 80, sodium stearyl fumarate, calcium phosphate dibasic, and mixtures thereof. The tablets may have a coating or film which may contain additional excipients such as iron oxide yellow, iron oxide black, iron oxide red polyvinyl alcohol, polyethylene glycol, talc, titanium dioxide, or mixtures thereof. One of skill in the art will be familiar with a variety of excipients and formulations that may be used to prepare desirable dosage forms with desired release characteristics and pharmacokinetic properties without undue experimentation.

In some embodiments, the tablets may contain venetoclax or a pharmaceutically acceptable salt thereof at an effective amount of between 10 mg and 100 mg. In particularly useful embodiments, the tablets have 10 mg, 50 mg, or 100 mg of effective venetoclax. Within the context of this invention, an effective amount refers to the amount of active venetoclax included within the dosage form, which accounts for the additional weight that a salt form may carry.

In view of the above description and the examples below, one of ordinary skilled in the art will be able to practice the invention as claimed without undue experimentation. The foregoing will be better understood with reference to the following examples that detail certain procedures for the preparation of molecules, compositions, and formulations according to the present invention. All references made to these examples are for the purposes of illustration. The following examples should not be considered exhaustive, but merely illustrative of only a few of the many aspects and embodiments contemplated by the present disclosure.

### EXAMPLES

### Example 1: Preparation of 2-chloro-4,4-dimethyl-2-oxocyclohexenecarbaldehyde

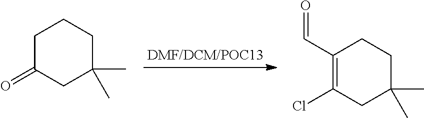

Dichloromethane (240 mL) and anhydrous dimethyl formamide (81 g) were cooled to 0-5° C. Phosphorous oxychloride (158 g) was added while maintaining the reaction mixture below 10° C. The reaction mass was stirred for 1 hour at 17-20° C., cooled to 0-5° C., 3,3-dimethylcyclohexanone (100 g) was added, and the reaction mass was refluxed for 3-5 hours at 45-50° C. The reaction mass was quenched in a mixture of 13.6% aqueous sodium acetate (320 mL), 12% brine solution (320 mL), and dichloromethane (320 mL) at 5-10° C. The reaction mixture was stirred at room temperature for 1 hour. The organic layer was separated and the aqueous layer was re-extracted with dichloromethane (240 mL). The combined organic layers were washed with 12% brine (380 mL) and 20% aqueous tripotassium phosphate (200 g). The dichloromethane layer was concentrated under vacuum, maintaining the temperature below 40° C., to get a brown colored oil (140 g, yield: 1.4 w/w).

### Example 2: Preparation of 2-(4-chlorophenyl)-4,4-dimethyl-2-oxocyclohex-1-enecarbaldehyde

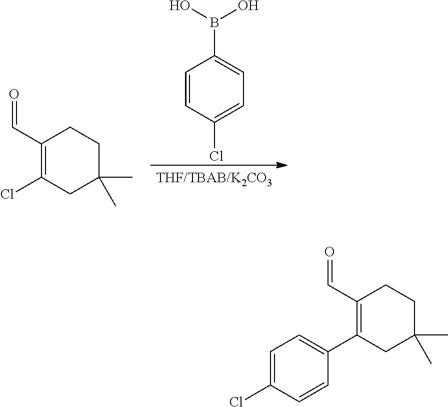 [SMILES: CC1(C)CCC(C=O)=C(Cl)C1 ; OB(O)c1ccc(Cl)cc1 ; CC1(C)CCC(C=O)=C(c2ccc(Cl)cc2)C1]

Tetrabutylammonium bromide (186.7 g) was added to a solution of 2-chloro-4,4-dimethyl-2-oxocyclohexenecarbaldehyde (100 g) and tetrahydrofuran (500 mL) at ambient temperature. An aqueous potassium carbonate solution (21.0%, 760 g (weight of solution)) and 4-chlorophenylboronic acid (95 g) were added and the solution was purged with nitrogen for 60-90 minutes. Palladium acetate (2 g) was added and the reaction mass was stirred for 3-4 hours at 30-35° C. then cooled to room temperature. Toluene (1 L), 10% sodium bicarbonate aqueous solution (500 mL), and 2% L-cysteine aqueous solution (500 mL) were added to the reaction mass, which was then stirred and allowed to settle. The toluene layer was separated and washed with 25% sodium chloride aqueous solution (1 L), then concentrated under vacuum at temperature 40-45° C. to get a brown oil. (170 g, Yield: 1.70 w/w).

### Example 3: Preparation of (2-(4-chlorophenyl)-4,4-dimethylcyclohex-1-enyl)methanol

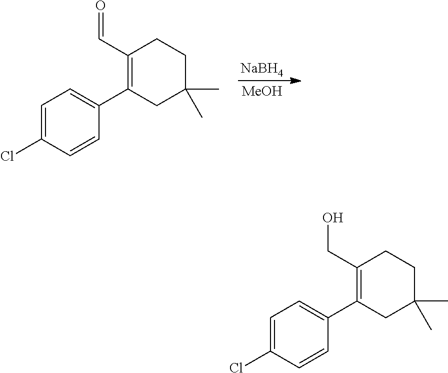

A mixture of 4′-chloro-5,5-dimethyl-3,4,5,6-tetrahydro-[1,1′-biphenyl]-2-carbaldehyde (100 g) and methanol (500 mL) was cooled to 0-5° C. Sodium borohydride (7.6 g) was added lot wise and the reaction was stirred for 2 hours at 0-5° C. The reaction was quenched by addition of water (500 mL) at 5-10° C. Hexane (500 mL) was added and the reaction mixture was stirred. The hexane layer was separated and concentration to provide a brown oil. (98 g, Yield: 0.98 w/w).

### Example 4: Preparation of a Compound of Formula 4

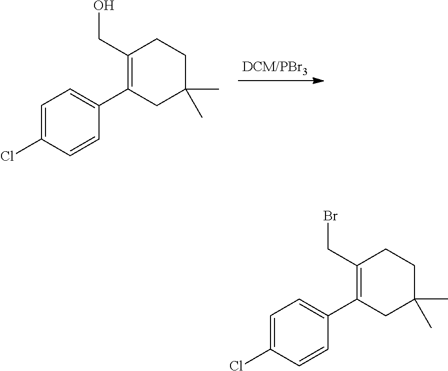 [SMILES: CC1(C)CCC(CO)=C(c2ccc(Cl)cc2)C1 ; CC1(C)CCC(CBr)=C(c2ccc(Cl)cc2)C1]

(2-(4-Chlorophenyl)-4,4-dimethylcyclohex-1-enyl)methanol (100 g) was treated with phosphorus tribromide (107 g) in dichloromethane (500 mL) at 0-5° C. for 2 hours, after which water (500 mL) was added at 10-12° C. The organic layer was separated and the aqueous layer was re-extracted with dichloromethane (500 mL). The combined dichloromethane layers were washed with 10% brine solution and concentration to provide a brown oil. (100 g, Yield: 1.0 w/w).

### Example 5: Preparation of a Compound of Formula 4

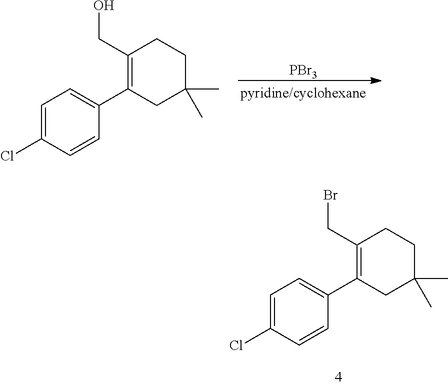 [SMILES: CC1(C)CCC(CO)=C(c2ccc(Cl)cc2)C1 ; "PBr3" ; CC1(C)CCC(CBr)=C(c2ccc(Cl)cc2)C1]

Phosphorus tribromide (107 g) was added to 1-hydroxy methyl-2-(4-chlorophenyl)-4,4-dimethylcyclohex-1-ene (100 g) and pyridine (6.4 mL) in cyclohexane (1000 mL) at 2-5° C. The reaction mixture was stirred for 2 hours at 2-5° C. Water (500 mL) was added at 10-12° C. and the aqueous and organic layers were separated. The cyclohexane layer was washed with 10% aqueous solution of NaHCO₃ (1000 mL) and 10% brine solution (1000 mL). The cyclohexane layer was concentrated, providing a white solid. (112.5 g, Yield=90%). ¹H NMR (500 MHz, CDCl₃) δ (ppm): 7.32 (d, J=8 Hz, 2H), 7.16 (d, J=8 Hz, 2H), 3.85 (s, 2H), 2.33 (t, J=7, 2H), 2.03 (s, 2H), 1.51 (t, J=7 Hz, 2H), 0.98 (s, 6H).

### Example 6: Preparation of methyl 2,4-difluorobenzoate

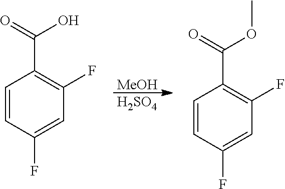

Sulphuric acid (97.4 g) was added to a mixture of 2,4-diflourobenzoic acid (100 g) and methanol (1 L) at ambient temperature. The reaction mixture was heated to 60° C. and maintained at that temperature for 8 hours. After cooling to room temperature, water (500 mL) was added. The reaction mixture was slowly added to chilled water (500 mL). The organic layer was separated and the aqueous layer was thrice extracted with dichloromethane (500 mL×3). The combined dichloromethane layers were washed with 10% sodium bicarbonate solution (200 mL) then concentrated under vacuum at temperature of not more than (NMT) 50-55° C. to give methyl 2,4-difluorobenzoate (88 g, Yield: 0.8 w/w).

### Example 7: Preparation of 4-cyano-tetrahydropyran-4-carboxylic Acid

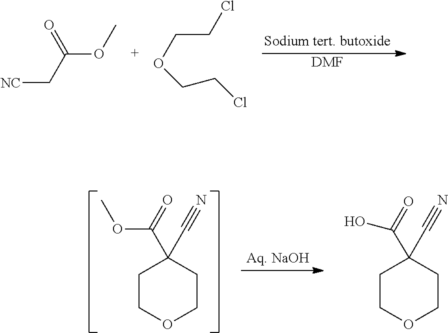

Cyanomethylacetate (100 g) was added to a mixture of sodium tert-butoxide (100 g) and dimethyl formamide (200 mL) at 0-5° C. The reaction mixture was warmed to 30° C., stirred for 3 hours, and was slowly added to bis(2-chloroethyl) ether (101 g). The reaction mixture was heated to 85° C. for 20 hours then quenched by adding water (500 mL). The pH was then adjusted to 9-11 by adding 60% aqueous sodium hydroxide solution (160 mL). The reaction mixture was stirred for 7 hours, ethyl acetate (300 mL) was added, and the organic and aqueous layers were separated. The aqueous layer was washed with ethyl acetate (300 mL). Concentrated HCl (175 mL) was added to the aqueous layer until the pH was between 2 and 3. Ethyl acetate (300 mL) was added and the reaction mixture was stirred. The aqueous layer was extracted with ethyl acetate (300 mL). The combined ethyl acetate layers were washed twice with water (300 mL×2) then concentrated under vacuum to get the desired product (90 g, Yield: 0.9 w/w).

### Example 8: Preparation of 4-cyano-tetrahydropyran

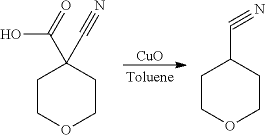

4-cyano-tetrahydropyran-4-carboxylic acid (100 g), copper (I) oxide (2.5 g), and toluene (1 L) were heated to 107° C. for 7 hours. After cooling to room temperature, the reaction mass was filtered through a Celite bed then concentrated at 60° C. to provide the desired crude product. Purification of the product was carried out by vacuum distillation at 60-65° C. (24 g, Yield: 0.24 w/w)

### Example 9: Preparation of 4-aminomethyltetrahydropuran

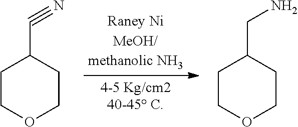 [SMILES: N#CC1CCOCC1 ; NCC1CCOCC1]

4-Cyano-tetrahydropuran (500 mL) in methanolic ammonia (200 mL) was hydrogenated in the presence of Raney nickel (10 g) under a pressure of 4 to 5 kg/cm² hydrogen gas for 12 hours at 45° C. After cooling, the reaction solution was filtered through a Celite bed. The reaction mixture was distilled at 55° C. to provide the desired product (60 g, Yield: 0.60 w/w).

### Example 10: Preparation of 4-chloro-3-nitrobenzene-1-sulfonamide

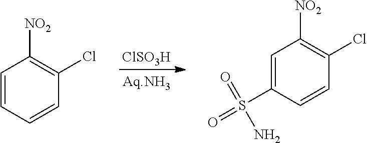

Chlorosulphonic acid (450 mL) was slowly added to 2-chloronitrobenzene (100 g). The reaction mass was heated to 100° C. and maintained at that temperature for 6 hours before cooling to ambient temperature and stirring for an additional 12 hours. The reaction mass was slowly poured into chilled aqueous ammonia (800 mL) and the reaction mixture was stirred for 3 hours at −10° C. The reaction mixture was then warmed to 23° C. and stirred for 2 h ours. The reaction mixture was then filtered and the obtained solid was washed three times with water (200 mL×3). The solid was dissolved in methanol (600 mL) at 60° C., charged with water (200 mL), and was stirred for 1 hour at 60° C. Again, the reaction mixture was charged with water (200 mL) and stirred for 1 hour at 60° C. Once again, the reaction mixture was charged with water (200 mL) and stirred for 1 hour. After cooling to room temperature and stirring for 1 hour, the reaction mixture was filtered, and the solid was washed with a 1:1 mixture of methanol and water (100 mL). The solid was dried under vacuum at 60° C. Crystallization of the residue with toluene gave the desired product (50 g, Yield: 0.5 w/w).

### Example 11: Preparation of a Compound of Formula 9

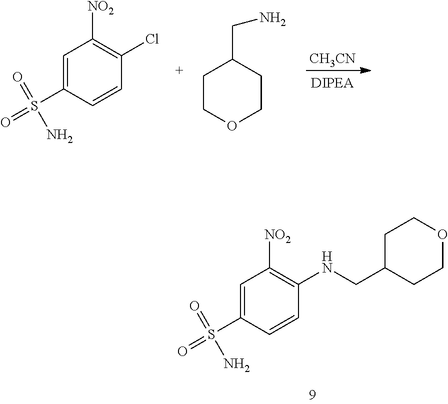

4-chloro-3-nitrobenzene-1-sulfonamide (100 g), 4-cyano-tetrahydropyran (97.4 mL), and N,N-diisopropylethylamine (160 mL) were heated in acetonitrile to 70° C. for 24 hours. After cooling to 55° C., the reaction mixture was distilled at 55° C. to remove solvent until two reaction mass volumes remained. Water (800 mL) was added to the reaction and stirred 1 hour. The reaction mixture was filtered and the solid was washed with water (200 mL), followed by acetonitrile (300 mL) and ethyl acetate (300 mL). The solid was dried under vacuum at 55° C. for 4 hours. (60 g, Yield: 0.6 w/w).

### Example 12: Preparation of 5-methoxy-7-azaindole

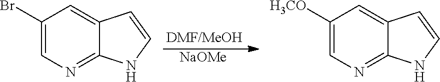 [SMILES: Brc1cnc2[nH]ccc2c1 ; COc1cnc2[nH]ccc2c1 ; "NaOMe"]

A mixture of 5-bromo-7-azaindole (100 g) and dimethyl formamide (800 mL) were cooled 5-10° C. Sodium methoxide (275 g), copper (I) iodide (194 g), and methanol (450 mL) were added and the temperature was raised to 95-100° C. and stirred for 2 hours. After cooling to 35° C., ethyl acetate (1 L) and aqueous ammonium chloride solution (200 g of ammonium chloride was dissolved in 600 mL water) were added, the reaction mixture was stirred for 4 hours and the reaction mass was filtered through a Celite bed. The filtrate was washed thrice with ethyl acetate (400 mL×3). Ethyl acetate (800 mL) and 30% aqueous ammonium chloride solution (1600 mL) were added, the reaction mixture was stirred, and the ethyl acetate layer was separated. The aqueous layer was extracted with ethyl acetate (400 mL) and the combined ethyl acetate layers were washed with 30% aqueous ammonium chloride solution (200 mL) until the blue colour disappeared. The ethyl acetate layer was concentrated under vacuum at 50° C. Toluene (200 mL) was charged to the residue at 50° C. and the reaction mass was stirred for 15 minutes. After cooling to 5-10° C., the reaction mixture was stirred for 2 hours then filtered. The obtained solid was washed with chilled toluene (50 mL) then dried under vacuum oven at 50° C. for 4 hours (58 g, Yield: 0.29 w/w).

### Example 13: Preparation of a Compound of Formula 7

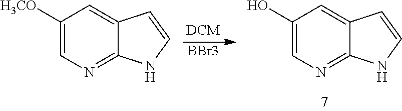

A solution of 5-methoxy-7-azaindole (75 g) and dichloromethane (1500 mL) to was cooled to 0-5° C. Boron tribromide (253 g) was added and the reaction mass was warmed to 27° C. and stirred for 4 hours. After cooling to 0-5° C., the reaction mass was quenched with methanol (225 mL). The reaction mass was stirred for 1 hour at 27° C. The organic layer was then concentrated under vacuum at 45° C. Ethyl acetate was added and the reaction mixture was stirred for 2 hours at room temperature before filtering. The obtained solid was washed with ethyl acetate (75 mL) then added to water (450 mL). The pH of the reaction mixture was adjusted to 7-8 using 10% aqueous sodium bicarbonate solution and then cooled to 0-5° C. The solution was filtered and the solid was washed with deionized water (75 mL). The solid was dried the solid under vacuum oven at 50° C. for 4 hours. (50 g, Yield: 0.66 w/w).

### Example 14: Preparation of a Compound of Formula 6 (Wherein R=Methyl and X═F)

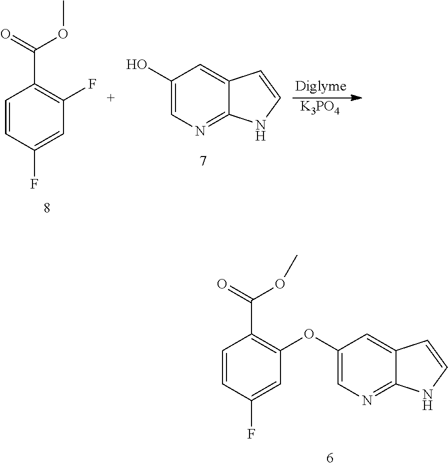

A mixture of formula 8 (R=methyl and X═F, 100 g), formula 7 (152 g), potassium phosphate (190 g), and diglyme were stirred at 110° C. for 20-22 hours. After cooling, the reaction mixture was filtered through a Celite bed and the filtrate was washed with diglyme (150 mL). Activated carbon (10 g) was charged and the mixture was stirred for 1 hour. The reaction mass was filtered through a Celite bed and the filtrate was washed with diglyme. Water (3000 mL) was added to the mother liquor and the mixture was stirred at 0-5° C. for 2 hours. The mixture was then filtered and the obtained solid was washed with water (450 mL) then dried at 60° C. under vacuum. Toluene was added and the mixture was stirred at 80° C. After cooling to 0-5° C., the reaction mixture stirred for 2 hours, filtered, and the obtained solid was washed with chilled toluene. The solid was suck dried then dried under vacuum (50 mm Hg) at 50° C. (80 g, Yield: 0.8 w/w).

### Example 15: Preparation of a Compound of Formula 5 (Wherein R=Methyl and X═F)

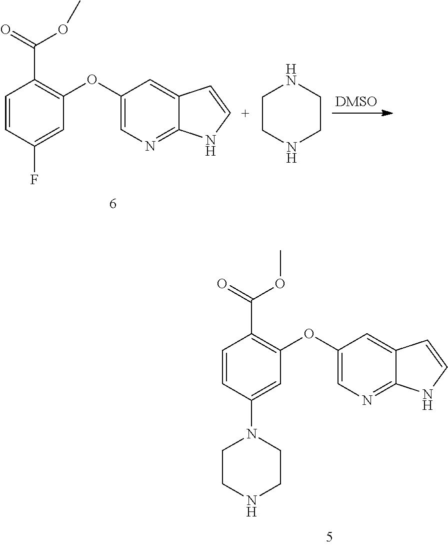 [SMILES: COC(=O)c1ccc(F)cc1Oc1cnc2c(c1)C=[C+]N2 ; C1CNCCN1 ; COC(=O)c1ccc(N2CCNCC2)cc1Oc1cnc2[nH]ccc2c1]

A mixture of formula 6 (R=methyl and X═F, 100 g) and piperazine (90 g) in dimethyl sulfoxide was heated to 90° C. for 2 hours. After cooling, water (1 L) and ethyl acetate (1 L) were added. The ethyl acetate layer was separated and the aqueous layer was re-extracted with ethyl acetate (200 mL). The combined organic layers were concentrated under vacuum at 40° C. Acetone (300 mL) and water (700 mL) were added to the residue at 40° C. The reaction mass was cooled to room temperature and stirred for 2 hours. The reaction mixture was filtered and the solid was washed with water (100 mL). The solid was dried under vacuum at 50° C. for 3 hours. (90 g, Yield: 0.9 w/w).

### Example 16:Preparation of a Compound of Formula 5a

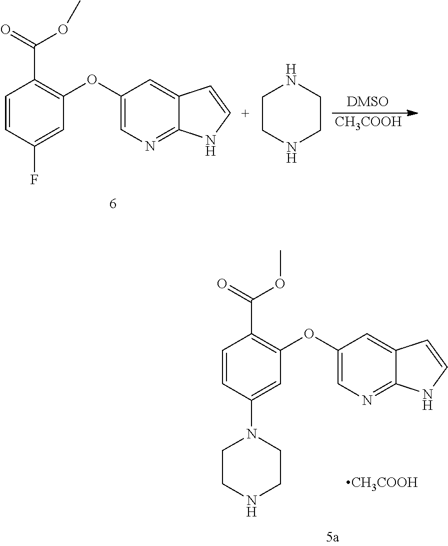

Formula 6 (R=methyl, 100 g) and piperazine (117.3 g) were combined in dimethyl sulfoxide (200 mL) and heated to 90° C. for 2 hours. The reaction mass was charged to a pre-cooled solution of methanol (100 mL) and water (2000 mL) at 10±5° C. and stirred for 4 hours at 10±5° C. The reaction mixture was filtered and the solid obtained was washed with water (200 mL). The wet cake was then dissolved in THF (600 mL) at 45±5° C. Activated carbon (10 g) was then charged to the solution which was stirred for 1 hour at room temperature. The solution was filtered and the bed was washed with THF (200 mL). Acetic acid (23 g) was then charged to the filtrate slowly after which it was stirred for 4 hours. The solution was filtered and the solid was washed with THF (200 mL). The solid was dried in a vacuum at 50° C. for 3 hours. (115 g, Yield=80%). ¹H NMR (500 MHz, DMSO-d6) δ (ppm): 11.64 (S, 1H), 8.00 (d, J=2.5 Hz, 1H), 7.76 (d, J=9 Hz, 1H), 7.47 (t, J=3 Hz, 1H), 7.43 (d, J=2.5 Hz, 1H), 6.77 (dd, J=2.5, 2.5 Hz, 1H), 6.37 (m, J=2, 2H, 2H), 3.65 (s, 3H), 3.10 (t, J=4.5 Hz, 4H), 2.7 (t, J=4.5 Hz, 4H), 1.90 (s, 3H).

### Example 17: Preparation of a Compound of Formula 3 (Wherein R=Methyl)

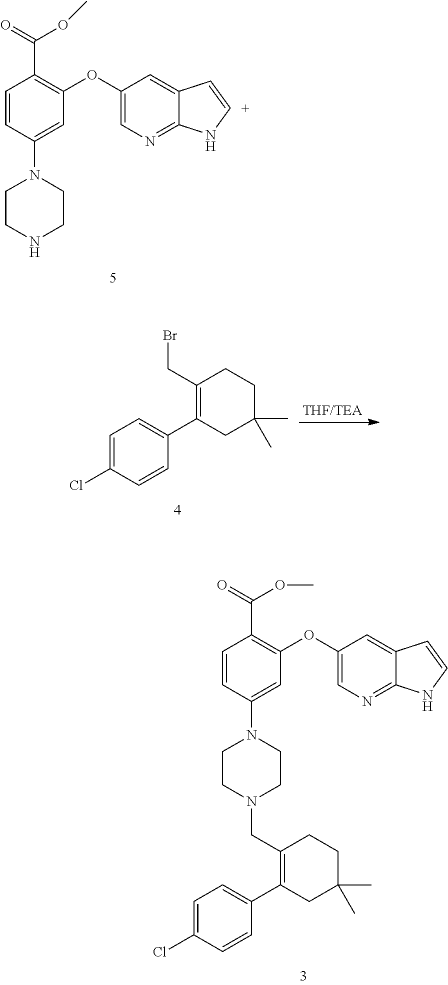 [SMILES: COC(=O)c1ccc(N2CCNCC2)cc1Oc1cnc2c(c1)C=[C+]N2 ; CC1(C)CCC(CBr)=C(c2ccc(Cl)cc2)C1 ; COC(=O)c1ccc(N2CCN(CC3=C(c4ccc(Cl)cc4)CC(C)(C)CC3)CC2)cc1Oc1cnc2[nH]ccc2c1]

A mixture of formula 5 (R=methyl, 100 g), formula 4 (117 g), and triethylamine (76.5 g) in tetrahydrofuran (1 L) were heated to 67° C. for 3 hours. After cooling, the reaction mixture was filtered through a Celitebed which was then washed with tetrahydrofuran (200 mL). The filtrate was concentrated under reduced pressure at 45° C. Toluene (800 mL) was added to the residue. After cooling, the toluene layer was washed with citric acid (600 mL) then heated to 60° C. for 10 minutes. It was then cooled and stirred at ambient temperature for 3 hours. The solution was filtered and the solid was washed with toluene (200 mL). The solid was dried under reduced pressure at 50 mm Hg at 50° C. and methanol (2600 mL) was added. The reaction mixture was heated to 60° C. for 1 hour. After cooling, the reaction mixture was stirred for 14 hours at ambient temperature. The solution was filtered to obtain a solid, which was washed with methanol (100 mL). The solid was dried under vacuum at 50° C. (127 g, Yield: 1.27 w/w).

### Example 18: Preparation of a Compound of Formula 3a

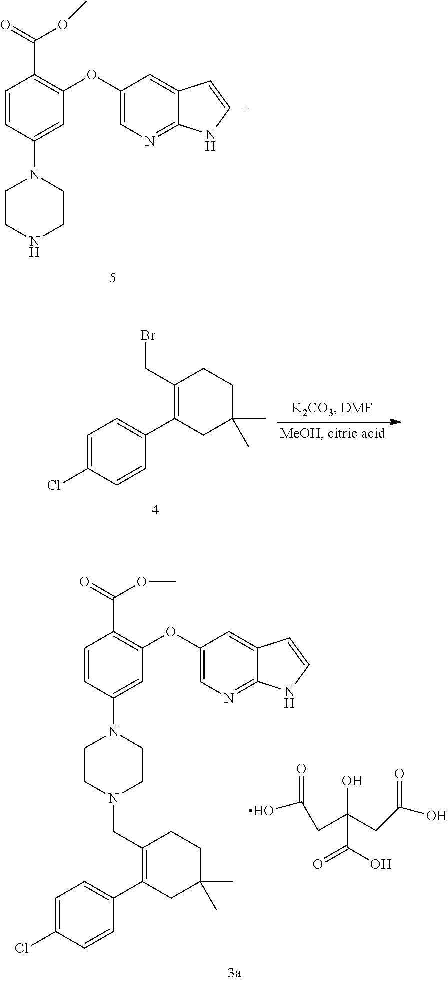

A mixture of formula 5 (R=methyl, 100 g), formula 4 (114 g), and K₂CO₃ (76.5 g) in dimethyl formamide (1000 mL) was stirred for 3 hours at room temperature. The reaction mass was charged with pre-cooled water (1000 mL) at 10±5° C. and stirred for 4 hours at room temperature. The solution was filtered and the solid was washed with water (200 mL). The wet cake was taken in methanol (1200 mL) and citric acid (69.9 g) was charged to the reaction mixture. The reaction mixture was then heated to reflux and stirred for 30 minutes. The reaction mixture was then filter through Hyflo under hot conditions and washed with methanol (200 mL). The reaction mixture was cooled slowly to room temperature then stirred for 2 hours. The solution was filtered and the solid was washed with methanol (200 mL) and dried under vacuum at 50° C. (170 g, Yield=90%). ¹H NMR (500 MHz, DMSO-d6) δ (ppm): 12.5 (br s, 2H), 11.64 (br s, 1H), 8.00 (d, J=2.5 Hz, 1H), 7.76 (d, J=9 Hz, 1H), 7.48 (t, J=3 Hz, 1H), 7.43 (d, J=2.5 Hz, 1H), 7.35 (d, J=8.5 Hz, 2H), 7.05 (d, J=8.5 Hz, 2H), 6.75 (dd, J=2.5, 2.5 Hz, 1H), 6.37 (m, J=2, 2H, 2H), 3.65 (s, 3H), 3.15 (s, 4H), 2.82 (s, 2H), 2.75 (d, J=15 Hz, 2H), 2.63 (d, J=15 Hz, 2H); 2.27 (s, 4H), 2.16 (s, 2H), 1.97 (s, 2H), 1.40 (t, J=6.5 Hz, 2H), 0.93 (s, 6H).

### Example 19: Preparation a Compound of Formula 2

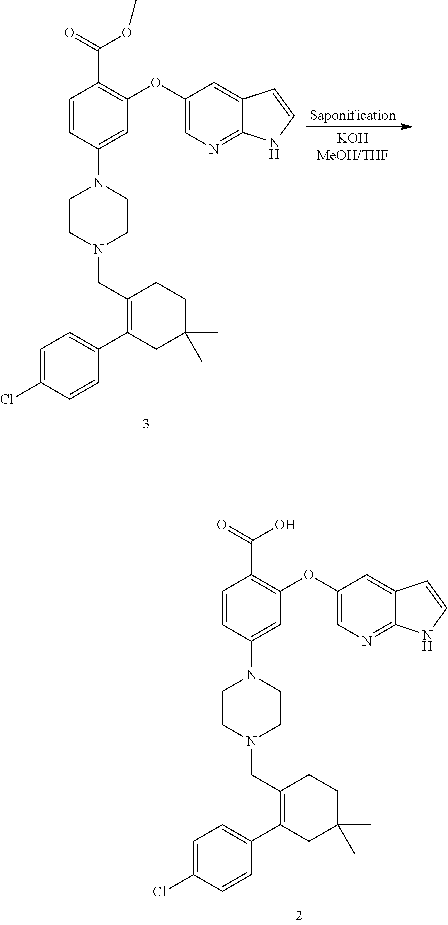 [SMILES: COC(=O)c1ccc(N2CCN(CC3=C(c4ccc(Cl)cc4)CC(C)(C)CC3)CC2)cc1Oc1cnc2[nH]ccc2c1 ; CC1(C)CCC(CN2CCN(c3ccc(C(=O)O)c(Oc4cnc5[nH]ccc5c4)c3)CC2)=C(c2ccc(Cl)cc2)C1]

A mixture of formula 3 (R=methyl, 100 g), methanol (500 mL), and potassium hydroxide (200 g) in tetrahydrofuran (500 mL) was stirred at 42° C. for 3 hours. The reaction mixture was distilled under reduced pressure at 45° C. and swapped the residue with acetonitrile (200 mL). Acetonitrile (1 L) was added in residue and stirred for 3 hours at 27° C. The reaction mixture was filtered and the solid was washed with acetonitrile (200 mL). Water (1 L) was added to wet the solid and the pH was adjusted to about 8 by adding aqueous 30% monopotassium phosphate solution (200 mL). The reaction mixture was filtered and the solid was washed with water (200 mL) and dried under vacuum at 50° C. (69 g, Yield: 0.69 w/w)

### Example 20: Preparation a Compound of Formula 2

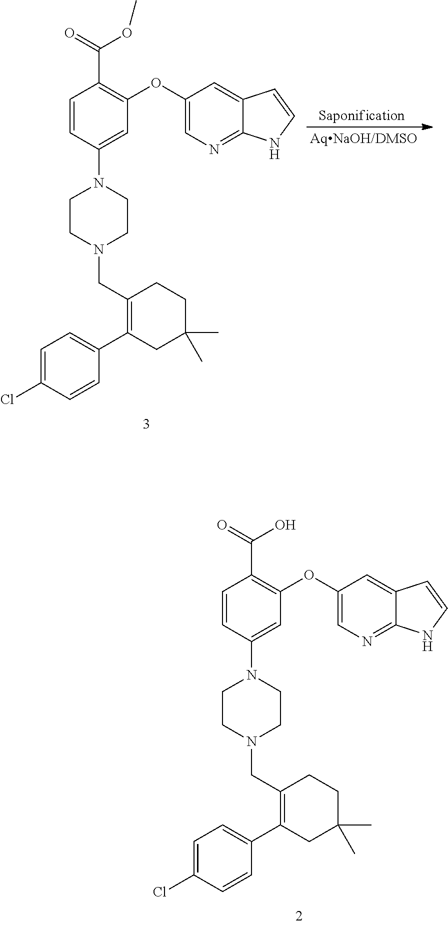 [SMILES: COC(=O)c1ccc(N2CCN(CC3=C(c4ccc(Cl)cc4)CC(C)(C)CC3)CC2)cc1Oc1cnc2[nH]ccc2c1 ; CC1(C)CCC(CN2CCN(c3ccc(C(=O)O)c(Oc4cnc5[nH]ccc5c4)c3)CC2)=C(c2ccc(Cl)cc2)C1]

Aqueous sodium hydroxide solution (70 g in 200 mL water) was charged slowly to a mixture of formula 3 (100 g) in dimethyl sulfoxide (1000 mL) and stirred for 3 hours at room temperature. Water (200 mL) was charged to the reaction mixture followed by slow addition of a solution of concentrated HCl (150 mL) and water (450 mL). The reaction mixture was stirred for 2 hours at room temperature. The solution was filtered and the solid was washed with water (400 mL). The solid was dried under vacuum at 50° C. (67 g, Yield=92%)

### Example 21: Preparation of a Compound of Formula 2

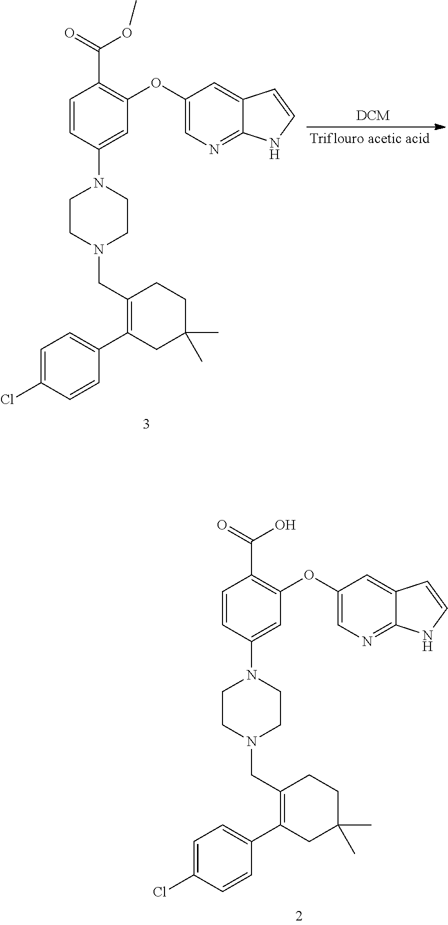 [SMILES: COC(=O)c1ccc(N2CCN(CC3=C(c4ccc(Cl)cc4)CC(C)(C)CC3)CC2)cc1Oc1cnc2[nH]ccc2c1 ; CC1(C)CCC(CN2CCN(c3ccc(C(=O)O)c(Oc4cnc5[nH]ccc5c4)c3)CC2)=C(c2ccc(Cl)cc2)C1]

A 1000 mL round bottom flask fitted with an overhead stirrer was charged with formula 3 (21 g, 33.48 mmol) and dichloromethane (420 mL). The reaction mixture was stirred at 20-25° C. to get a clear solution to which trifluoroacetic acid (31 mL) was added drop wise over a period of 30 minutes and then held at a temperature of 20-25° C. for 16 hours. The progress of the reaction was monitored by TLC. After completion of the reaction, the reaction was quenched into a sodium bicarbonate solution (63 g of sodium bicarbonate was dissolved into 1000 mL of water) at a pH 6 to 7. The reaction mixture was extracted three times with dichloromethane (3×210 mL) and the combine organic layers were washed with 210 mL of water. The combined organic layer was dried over sodium sulfate and the organic layer was concentrated on a rotatory evaporator. Methyl tert-butyl ether (105 mL) was added to the concentrated mass and heated to 45-50° C. to get a clear solution. N-heptane (210 mL) was added and the reaction mass was cooled 25-30° C. and stirred for 1 hour. The reaction mass was filtered and the cake was washed with 20 mL heptane and dried under reduced pressure at 50° C. Yield: 16 g; 83.7%)

### Example 22: Preparation of a Compound of Formula 2

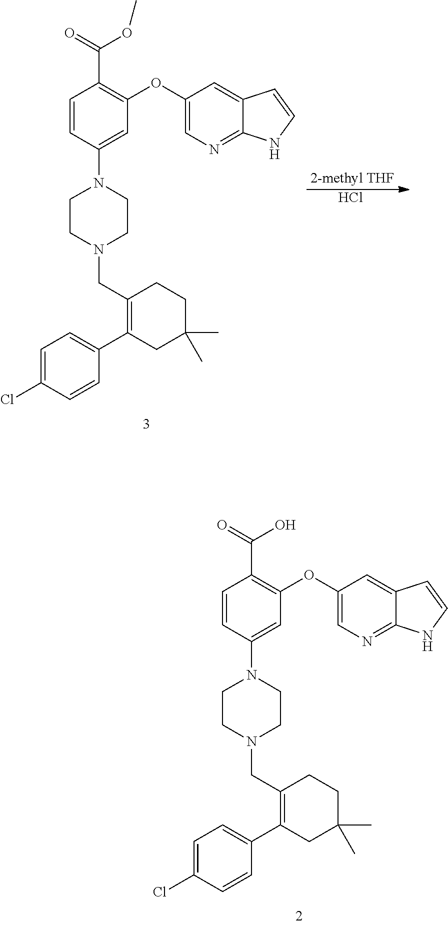 [SMILES: COC(=O)c1ccc(N2CCN(CC3=C(c4ccc(Cl)cc4)CC(C)(C)CC3)CC2)cc1Oc1cnc2[nH]ccc2c1 ; Cl ; CC1(C)CCC(CN2CCN(c3ccc(C(=O)O)c(Oc4cnc5[nH]ccc5c4)c3)CC2)=C(c2ccc(Cl)cc2)C1]

A 50 mL round bottom flask fitted with an overhead stirrer was charged with formula 3 (2.8 g) and 2-methyl tetrahydrofuran (17 mL) at 20-25° C. The reaction mixture was stirred at 20-25° C. to get a clear solution, to which 17 mL 6N HCl was added drop wise over a period of 10 minutes and then held at temperature 20-25° C. for 16 hours. The progress of the reaction was monitored by TLC. After completion of the reaction, the reaction mass was concentrated on a rotatory at reduced pressure and was added 28 mL dichloromethane and stirred for 30 minutes. The organic layers were separated then washed with brine (28 mL) and concentrated under reduced pressure. The product was purified by column chromatography on silica gel (eluent methanol:dichloromethane (9:1) mixture). The product fractions were combined and concentrated on a rotatory evaporator under reduced pressure at a temperature of 50-60° C. to get 1.5 g of the titled compound. Yield: 0.53 w/w (Molar: 58.3%)

### Example 23: Preparation of Venetoclax

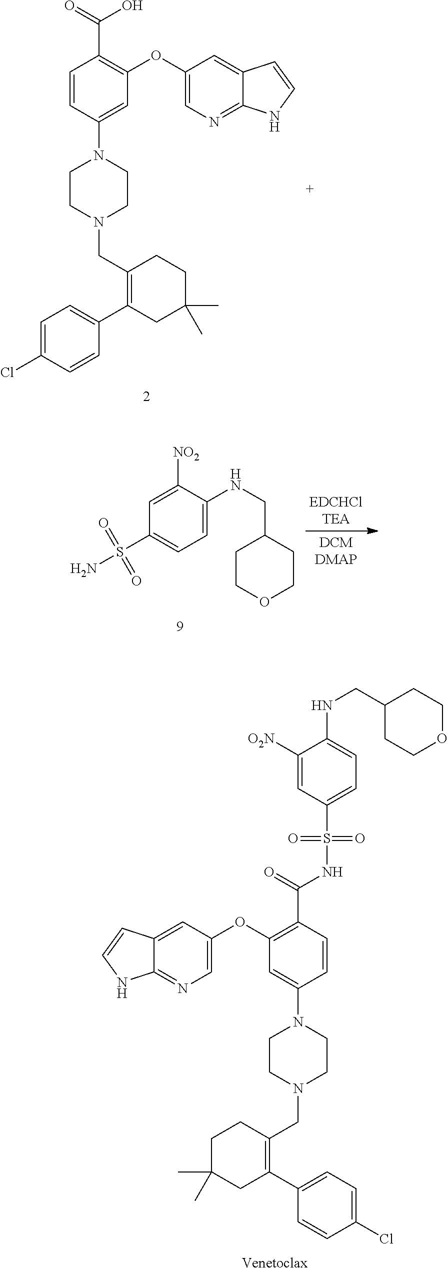 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Formula 2 (100 g) and triethylamine (36 g) were stirred in dichloromethane (500 mL). In another flask, formula 9 (40 g), 4-dimethylaminopyridine (42.8 g) and 1-ethyl-3-(3-dimethylaminopropyl)carbodiimide (42.8 g) in dichloromethane (1.2 L) were stirred at ambient temperature. To this second solution, approximately 70% of the formula 2 solution was added over 6 hour and then stirred further for another 2 hours. After 2 hours, 4-dimethylaminopyridine (21.4) and 1-ethyl-3-(3-dimethylaminopropyl) carbodiimide (23.6 g) were added. The remaining amount of the formula 2 solution was then added over 2 hours. The reaction was monitored by TLC. 10% aqueous acetic acid aqueous (750 mL) was added to the reaction mixture which was then stirred for 30 minutes. The dichloromethane layer was separated and washed with 5% aqueous sodium bicarbonate solution (750 mL) then 5% sodium chloride solution (750 mL). The dichloromethane layer was then concentrated under vacuum at 40° C. The obtained crude material was purified in a mixture of methanol (700 mL) and ethyl acetate (700 mL). The mixture was filtered and the solid was washed with mixture of methanol and ethyl acetate (200 mL). The solid was dried under vacuum to obtain venetoclax. (70 g, Yield: 0.7 w/w)

### Example 24: Preparation of Venetoclax

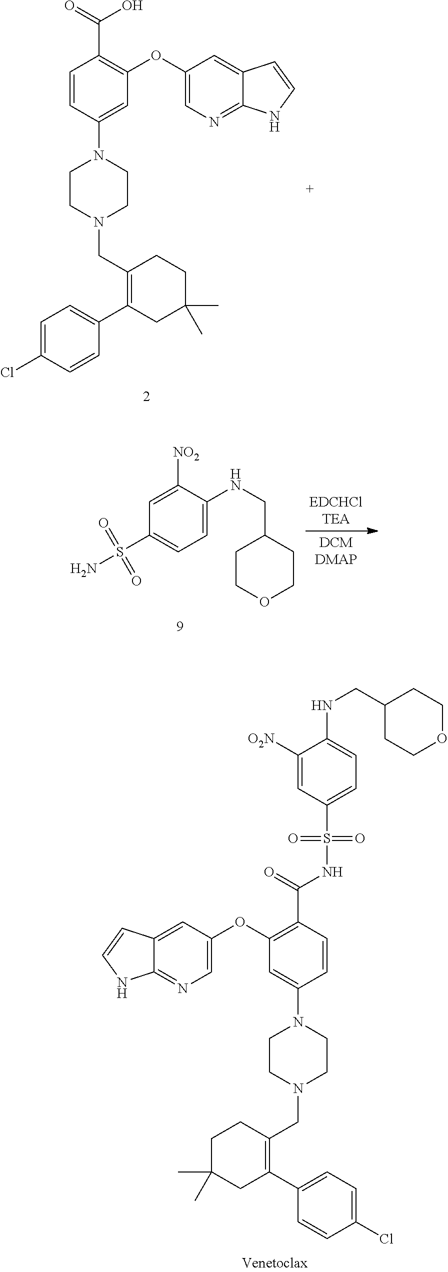 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Formula 2 (100 g) and triethylamine (35.2 g) was stirred in dichloromethane (500 mL). In another flask, formula 9 (46.9 g), 4-dimethylaminopyridine (42.68 g), 1-ethyl-3-(3-dimethylaminopropyl) carbodiimide (46.96 g) in dichloromethane (1.2 L) was stirred. The solution of formula-2 was added slowly to the solution of formula 9 at ambient temperature and the reaction was stirred for 12 hours. The organic layer was washed with 10% acetic acid solution (750 mL) twice, followed by 5% aqueous NaHCO₃ (750 mL) and 5% aqueous NaCl (750 mL). The dichloromethane layer was concentrated under vacuum at 40° C. Dichloromethane (900 mL) was added and the reaction mixture was heated to 38° C. Methanol (100 mL) and ethyl acetate (800 mL) were added at 38° C. The reaction mass was cooled to 27±3° C., stirred for 2 hours, and filtered. The solid was washed with a mixture of dichloromethane (150 mL) and ethyl acetate (150 mL). The solid was dried under vacuum at 60±5° C. for 4 hours. Dry weight: 76 g (Yield=50%).

### Example 25: Preparation of Venetoclax

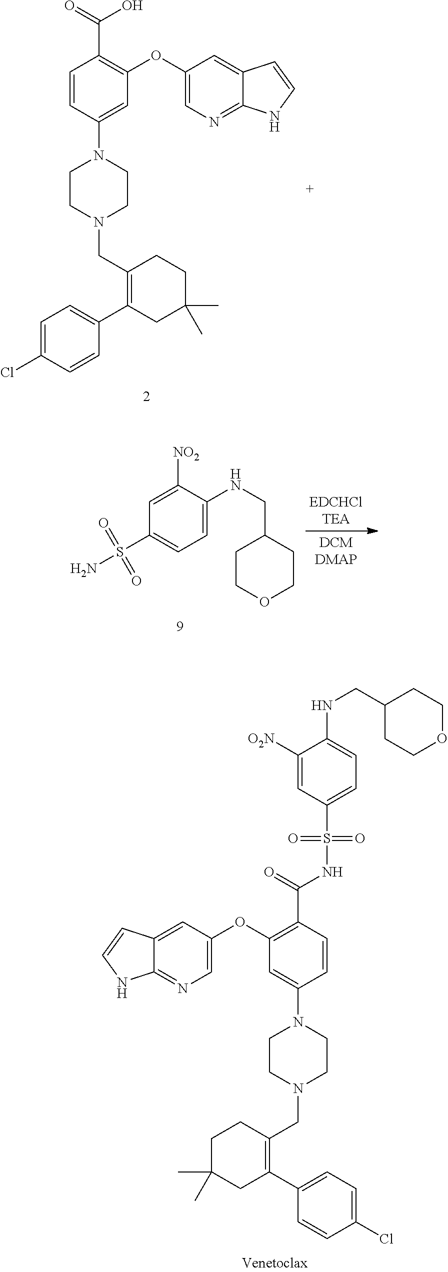 [SMILES: CC1(C)CCC(CN2CCN(c3ccc(C(=O)O)c(Oc4cnc5[nH]ccc5c4)c3)CC2)=C(c2ccc(Cl)cc2)C1 ; NS(=O)(=O)c1ccc(NCC2CCOCC2)c([N+](=O)[O-])c1 ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

In a clean, dry, four-necked 250 mL round bottom flask fitted with an overhead stirrer and reflux condenser, formula 2 (10 g, 17.5 mmol) and 3.55 g (35.1 mmol) of triethyl amine (TEA) were charged, followed by addition of 48 mL of dichloromethane at 20-25° C. The reaction mixture was stirred to get a clear solution. In another four-necked 250 mL round bottom flask fitted with an overhead stirrer and reflux condenser, Formula 9 (4.7 g, 14.9 mmol), 4-dimethylamino pyridine (4.2 g, 34.4 mmol), and N-(3-dimethylaminopropyl)-N′-ethylcarbodiimide hydrochloride (4.28 g, 22.3 mmol) were charged, followed by addition of dichloromethane (120 mL). The reaction mass was stirred to get a suspension. The solution of formula 2 was then added drop wise to the suspension of formula 9 over a period of 10 minutes at a temperature 20-25° C., after which the reaction mixture was stirred for 15 hours. The progress of the reaction was monitored by TLC. After completion of the reaction, N,N′-dimethylethylenediamine was added and the mixture was heated to 30-35° C. for 30 min. The reaction mass was washed with 2×74 mL of 10% aqueous acetic acid. The aqueous and organic layers were separated and a mixture of dichloromethane (30 mL) dichloromethane and methanol (5 mL) was added. The organic layer was washed with 2×74 mL 5% aqueous sodium bicarbonate solution and concentrated under reduced pressure to get crude venetoclax. The crude product was purified by column chromatography using silica gel (mobile phase dichloromethane:methanol (98:2)) to get amorphous venetoclax. Yield: 10 g; w/w: 0.53 w/w (Molar: 58.3%)

### Example 24: Preparation of Amorphous Venetoclax

In a clean, dry, four-necked 25 mL flask fitted with a magnetic stirring and reflux condenser, 1.24 g of venetoclax was combined with 12 mL acetonitrile and heated to 55-60° C. to get a clear solution. The solution was gradually cooled to 8-10° C. and maintained for 1 hour. The reaction mass was filtered, the cake was washed with 2 mL acetonitrile, and the washed cake was dried under reduced pressure at 40-45° C. Yield: 0.7 g; 56.4%, PXRD analysis: amorphous

We claim:

1. A process for the preparation of venetoclax comprising the steps of:
a) condensing formula 5 with formula 4 in the presence of a base to obtain formula 3; and 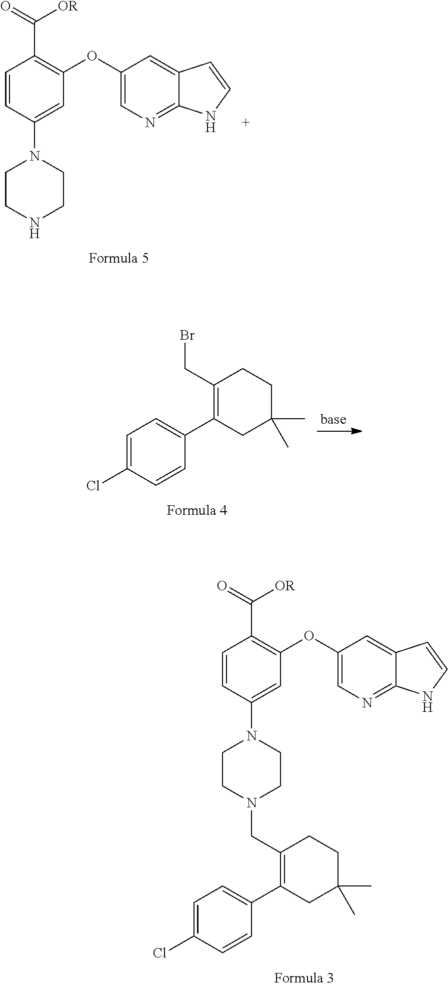
b) hydrolyzing formula 3.

2. The process according to claim 1, wherein formula 5 and formula 3 are pharmaceutically acceptable salts of formula 5 and formula 3, respectively.

3. The process according to claim 1, further comprising converting formula 3 to venetoclax.

4. The process according to claim 3, further comprising converting venetoclax to a pharmaceutically acceptable salt of venetoclax.

5. The process according to claim 1, wherein the base is selected from the group consisting of sodium hydroxide, potassium hydroxide, potassium carbonate, sodium carbonate, ammonium hydroxide, potassium phosphate, and mixtures thereof.

6. The process according to claim 1, wherein the solvent is an ether.

7. The process according to claim 5, wherein the ether is selected from the group consisting of tetrahydrofuran, diglyme, diethyl ether, diisopropyl ether, dimethyl sulfoxide, dimethyl formamide, and mixtures thereof.

8. (canceled)

9. The process according to claim 1, wherein a process for the preparation of formula 4 comprising reacting 1-hydroxymethyl-2-(4-chlorophenyl)-4,4-dimethylcyclohex-1-ene with phosphorus tribromide in the presence of a solvent.

10. The process according to claim 9, wherein the solvent is a hydrocarbon.

11. The process according to claim 10, wherein the hydrocarbon is selected from the group consisting of hexane, heptane, cyclohexane, methyl cyclohexane, and mixtures thereof.

12. An acetate salt of formula 5, wherein R=methyl, characterized by a PXRD pattern having substantial peaks at 20 angles of 11.73, 13.14, 14.69, and 26.55±0.2°.

13. The acetate salt of formula 5 according to claim 12, further characterized by the PXRD pattern in FIG. 2.

14. The acetate salt according to claim 12, further characterized by a PXRD pattern having substantial peaks at 2Θ angles of 7.49, 11.73, 12.75, 13.14, 14.69, 15.27, 16.10, 16.35, 17.27, 18.00, 18.89, 19.21, 19.86, 20.27, 21.04, 22.06, 22.36, 23.26, 23.51, 23.91, 24.36, 25.02, 25.70, 26.55, 27.30, 28.53, 29.25, 29.71, 30.49, 30.91, 31.60, 32.05, 32.89, 34.18, 34.46, 35.58, 36.28, 37.89, 38.29, 39.63, 41.32, 42.54, 43.45, 44.05, 44.92, 45.59, 48.04, 48.37, and 48.96±0.2°.

15. (canceled)

16. (canceled)

17. (canceled)

18. A citrate salt of formula 3, wherein R=methyl, characterized by a PXRD pattern having substantial peaks at 20 angles of 19.94, 15.88, 17.55, and 20.26±0.2°,

19. The citrate salt according to claim 18, further characterized by the PXRD pattern in FIG. 1.

20. The citrate salt according to claim 18, further characterized by a PXRD pattern having substantial peaks at 2Θ angles of 6.37, 8.05, 11.52, 12.54, 13.16, 15.88, 16.43, 17.55, 19.20, 19.94, 20.26, 22.22, 22.92, 23.33, 27.02 and 30.21±0.2°.

21. (canceled)

22. (canceled)

23. (canceled)

24. A process for the preparation of amorphous venetoclax, comprising the steps of:
a) dissolving venetoclax in a solvent to form a solution;
b) cooling the solution; and
c) isolating amorphous venetoclax.

25. The process according to claim 24, wherein the dissolving step is done at an elevated temperature.

26. The process according to claim 24, wherein the solution is cooled to 0° C.-15° C.

27. The process according to claim 24, wherein the solvent is selected from the group consisting of acetonitrile, acetone, methyl isobutyl ketone, and mixtures thereof.
